(19) United States
(12) Patent Application Publication
(10) Pub. No.: US 2020/0308546 A1
(43) Pub. Date: Oct. 1, 2020

(54) ENGINEERED CARDIOMYOCYTES AND USES THREOF

(71) Applicant: THE J. DAVID GLADSTONE INSTITUTES, A TESTAMENTARY TRUST ESTABLISHED UNDER THE WILL OF WILL OF J. DAV, San Francisco, CA (US)

(72) Inventors: Deepak SRIVASTAVA, San Francisco, CA (US); Yen-Sin ANG, San Francisco, CA (US)

(73) Assignee: The J. David Gladstone Institutes, A Testamentary Trust Established Under the Will of Will of J. Dav, San Francisco, CA (US)

(21) Appl. No.: 16/311,859

(22) PCT Filed: Jun. 27, 2017
(86) PCT No.: PCT/US2017/039576

§ 371 Date: Dec. 20, 2018

Related U.S. Application Data
(60) Provisional application No. 62/354,937, filed on Jun. 27, 2016.

(51) Int. Cl. C12N 5/077; G01N 33/50

(52) U.S. Cl. CPC C12N 5/0657; G01N 33/5044; C12N 2506/45; C12N 2503/00; C12N 2501/999

(57) ABSTRACT

The present disclosure provides the development of engineered cardiomyocytes having mutations in transcription factor involved in vivo with cardiac development and/or function. These cell populations comprise mutations that are associated with deleterious effects in vivo in mammals. The mutations of the engineered cardiomyocytes of the disclosure thus are rationally designed based on demonstrated physiological effects in mammals, e.g., mice or humans.

### CROSS-REFERENCE

This application claims benefit of U.S. Provisional Patent Application No. 62/354,937, filed Jun. 27, 2016, which is incorporated herein by reference in its entirety.

### FEDERALLY SPONSORED RESEARCH AND DEVELOPMENT

This invention was made with Government support under grant number HL089707 awarded by the National Institutes of Health. The Government has certain rights in the invention.

### FIELD OF THE INVENTION

The present invention relates generally to the fields of cell biology, pluripotent stem cells, and cell differentiation. The invention discloses populations of neural precursor cells and therapeutic uses thereof.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Cell identity and function require accurate transcription factor localization. Numerous transcriptional and epigenetic regulators of the cardiac cell-fate machinery have emerged (McCulley and Black, 2012; Srivastava, 2006), but most identified transcription factors are not exclusively expressed in cardiac tissues. Instead, they physically and functionally interact to co-regulate cardiac development and function (He et al., 201-1; Luna-Zurita et al.). However, it remains unknown what specific disruption of the interaction of gene regulatory networks contributes to human disease.

Clinical management of conditions, diseases and injuries of the cardiovascular system remain an area of significant unmet clinical need. Thus, there remains a pressing need for improved and effective treatments and methods of identifying therapeutic agents to address cardiovascular disease.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides the development of engineered cardiomyocytes having mutations in transcription factors and/or signaling pathways that disrupt cardiac development and/or function. These cell populations comprise mutations that are associated with deleterious cardiac effects in vivo in mammals. The mutations of the engineered cardiomyocytes of the disclosure are rationally designed based on demonstrated physiological effects in mammals, e.g., mice or humans.

In some aspects, the disclosure provides induced pluripotent stem cell-derived engineered cardiomyocytes (iPSC-cardiomyocytes) having one or more GATA4 mutations as described herein. In other aspects, the disclosure provides pluripotent stem cell-derived engineered cardiomyocytes (pSC-cardiomyocytes) having one or more GATA4 mutations as described herein. In specific aspects these cells are mammalian, e.g., rodent or human. These populations of cells are useful to screen candidate agents for their effect on cardiac development and/or function.

In other aspects, the disclosure provides induced pluripotent stem cell-derived engineered cardiomyocytes (iPSC-cardiomyocytes) having one or more TBX5 mutations as described herein.. In specific aspects these cells are mammalian, e.g., rodent or human. In other aspects, the disclosure provides pluripotent stem cell-derived engineered cardiomyocytes (pSC-cardiomyocytes) having one or more TBX5 mutations as described herein. These populations of cells are useful to screen candidate agents for their effect on cardiac development and/or function.

In specific aspects, the disclosure provides induced pluripotent stem cell-derived engineered cardiomyocytes (iPSC-cardiomyocytes) having one or more mutations in the PI3K signaling pathway as described herein. In other aspects, the disclosure provides pluripotent stem cell-derived engineered cardiomyocytes (pSC-cardiomyocytes) having one or more mutations in a molecule of the PI3K signaling pathway as described herein. In specific aspects these cells are mammalian, e.g., rodent or human. These populations of cells are useful to screen candidate agents for their effect on cardiac development and/or function.

In certain aspects, the present invention relates to a method for screening a therapeutic drug candidate using engineered cardiomyocyte populations comprising one or more of the physiologically relevant mutations as disclosed herein. These methods of screening with candidate agents are also useful for assessing the cardiotoxicity of agents.

Accordingly, the disclosure provides a method for screening a candidate agent for therapeutic use, comprising the steps of: contacting at least one engineered cardiomyocyte comprising a mutation in GATA4 with a candidate agent in vitro and determining the effect of the candidate agent based on changes in at least one cellular property of the engineered cardiomyocyte compared to a control comprising at least one engineered cardiomyocyte absent the mutation, where the candidate agent is identified as a viable candidate agent for therapeutic use if it has a positive physiological effect on the cellular property of the mutant cell as compared to the cell absent the mutation.

The disclosure further provides a method for screening a candidate agent for therapeutic use, comprising the steps of: contacting at least one engineered cardiomyocyte comprising a mutation in TBX5 with a candidate agent in vitro and determining the effect of the candidate agent based on changes in at least one cellular property of the engineered cardiomyocyte compared to a control comprising at least one engineered cardiomyocyte absent the mutation, where the candidate agent is identified as a viable candidate agent for therapeutic use if it has a positive physiological effect on the cellular property of the mutant cell as compared to the cell absent the mutation.

The disclosure further provides a method for screening a candidate agent for therapeutic use, comprising the steps of: contacting at least one engineered cardiomyocyte comprising a mutation in a gene of the phosphoinositide 3-kinase (PI3K) pathway with a candidate agent in vitro and determining the effect of the candidate agent based on changes in at least one cellular property of the engineered cardiomyocyte compared to a control comprising at least one engineered cardiomyocyte absent the mutation, where the candidate agent is identified as a viable candidate agent for therapeutic use if it has a positive physiological effect on the cellular property of the mutant cell as compared to the cell absent the mutation.

The engineered cardiomyocyte cell populations of the disclosure can also be used for assessing the cardiotoxicity of an agent. For example, an agent may be considered cardiotoxic if the agent excessively exerts a negative effect on at least one engineered cardiomyocyte compared to the control. An agent may also be considered cardiotoxic if it increases or decreases at least one electrophysiological property to such an extent that it cannot be considered safe for use. For example, an agent that increases the field potential duration (FPD), minimum field potential (Fpmin), conduction velocity, and/or the beating frequency excessively is unlikely to be safe. Alternatively, an agent that decreases the field potential duration (FPD), minimum field potential, (Fpmin), conduction velocity and/or the beating frequency, excessively (for example, almost to a flat line) is unlikely to be safe. Further, an agent may also be considered cardiotoxic if it causes a change in the regularity of beating rhythm, for example an irregular beating rhythm. The cardiotoxicity of agents with therapeutic applications other than cardiovascular therapy (i.e. non-cardiovascular agents) may also be assessed.

In other aspects, the present invention provides a method for predicting risk of and/or predisposition to cardiomyopathy in a subject following administration of a candidate agent, comprising: providing at least one engineered cardiomyocyte derived from the subject, contacting the at least one engineered cardiomyocyte with a candidate agent, and determining a risk for the subject of cardiomyopathy following administration of the candidate agent.

Further, the method may be used for selecting a dosage and/or dosage range of the agent. The induced pluripotent stem cell-derived engineered cardiomyocyte is a useful model to select for suitable dosage and/or dosage ranges. For example, the effect of various dosages of the agent is compared with said control and a suitable dosage or dosage range of the agent capable of exerting said effect may be selected. In particular, a suitable dosage and/or dosage range would be one exerts an effect likely to be therapeutically effective with minimal cardiotoxicity.

The disclosure also provides a method for screening an agent as a candidate agent for therapeutic use, comprising the steps of: contacting at least one engineered cardiomyocyte comprising a mutation in GATA4 with a candidate agent in vitro at different dosages; and determining the effect of the candidate agent based on changes in at least one cellular property of the engineered cardiomyocyte contacted with different dosages of the agent compared to a control comprising at least one engineered cardiomyocyte absent the agent, where the therapeutically effective dosage of the candidate agent for therapeutic is determined by identifying the dosage having a positive physiological effect on the cellular property of the mutant cell as compared to the cell absent the agent.

The disclosure also provides a method for screening an agent as a candidate agent for therapeutic use, comprising the steps of: contacting at least one engineered cardiomyocyte comprising a mutation in TBX5 with a candidate agent in vitro at different dosages; and determining the effect of the candidate agent based on changes in at least one cellular property of the engineered cardiomyocyte contacted with different dosages of the agent compared to a control comprising at least one engineered cardiomyocyte absent the agent, where the therapeutically effective dosage of the candidate agent for therapeutic is determined by identifying the dosage having a positive physiological effect on the cellular property of the mutant cell as compared to the cell absent the agent.

The disclosure also provides a method for screening an agent as a candidate agent for therapeutic use, comprising the steps of: contacting at least one engineered cardiomyocyte comprising a mutation in a gene of the PI3K pathway with a candidate agent in vitro at different dosages; and determining the effect of the candidate agent based on changes in at least one cellular property of the engineered cardiomyocyte contacted with different dosages of the agent compared to a control comprising at least one engineered cardiomyocyte absent the agent, where the therapeutically effective dosage of the candidate agent for therapeutic is determined by identifying the dosage having a positive physiological effect on the cellular property of the mutant cell as compared to the cell absent the agent.

Induced pluripotent stem cells (iPSC) may be generated from adult somatic cells by any method, including but not limited to reprogramming to the embryonic state by viral or non-viral based methods. These iPSCs resemble embryonic stem cells in self renewal capacities and differentiation potential to various cell types including engineered cardiomyocytes. For example, iPSCs may further be differentiated into cardiomyocyte-like cells by any method. Such induced pluripotent stem cell (iPSC)-derived engineered cardiomyocytes (iPSC-cardiomyocytes) were found to be a useful model for screening agents as drug candidates. According to a particular aspect, human induced pluripotent stem cells (hiPSCs) may be used to derive human induced pluripotent stem cell-derived engineered cardiomyocytes (hiPSC-cardiomyocytes).

The present disclosure also relates to Induced pluripotent stem cell (iPSC) derived engineered cardiomyocyte(s) for use in screening an agent as a therapeutic drug candidate. In certain aspects, the assays of the disclosure for screening agents customized to an individual subject. Accordingly, engineered cardiomyocytes derived from stem cells (e.g., iPSCs) from the subject may be used to screen a candidate therapeutic agent for the subject.

Engineered cardiomyocytes can be produced using any method available in the art, as will be apparent to one skilled in the art upon reading the present disclosure. In one aspect, the cardiomyocytes are produced from mammalian pluripotent stem cells, e.g., rodent or human pluripotent stem cells. For example, cells may be generated by a process of in vitro differentiation of pluripotent stems cells (e.g., human stem cells) comprising the mutation of interest to engineered cardiomyocytes. In a another example, induced pluripotent stem cells may be generated by a process of reprogramming of somatic cells isolated from normal or diseased mammalian subjects, and then differentiated in vitro to engineered cardiomyocytes. The present disclosure also relates to induced pluripotent stem cell (iPSC) derived engineered cardiomyocyte(s) for use in screening an agent as a drug candidate

In certain aspects, the disclosure provides iPS-derived engineered cardiomyocytes from subjects with a mutation that disrupts GATA4 and/or TBX5 binding at super enhancer elements associated with genes required for heart development and/or muscle contraction. These cells display an impaired contractility, calcium handling and metabolic activity as shown herein.

In specific aspects, the disclosure provides iPS-derived engineered cardiomyocytes from subjects with a GATA4-G296S mutation. These cells display an impaired contractility, calcium handling and metabolic activity. The GATA4-G296S mutation disrupted recruitment of TBX5, another cardiogenic transcription factor shown to co-occupy cardiac enhancers with GATA4. GATA4-G296S mutation led to GATA4 and TBX5 mislocalization to non-cardiac genes, and enhanced open chromatin states at these loci, particularly at endothelial/endocardial promoters, Correspondingly, GATA4 mutants failed to silence endothelial/endocardial gene expression as part of a broader dysregulation of cell identity.

These aspects and other features and advantages of the invention are described below in more detail. Those skilled in the art will recognize, or be able to ascertain using no more than routine experimentation, many equivalents to the specific embodiments of the invention described herein. Such equivalents are intended to be encompassed by the following claims.

### BRIEF DESCRIPTION OF THE FIGURES

FIG. 1 is a schematic showing the GATA 4 mutation distribution in four subjects harboring the GATA4 G296S mutation, and four family members without the mutation. Females are shown in circles and males in squares. A bolded border denotes CRISPR-corrected iPS lines. WT, wildtype familial control; G296S, patients with this mutation in GATA4; cmy, cardiomyopathy; ASD, atrial septal defect; VSO, ventricular septal detect AVSD, atrioventricular septal defect; PS, pulmonary valve stenosis. The bottom shows a schematic of the GATA4 protein domains. TAD, transactivation domain; ZF, zinc-finger domain; NLS, nuclear localization signal.

FIG. 2 is a chart summarizing the donor status and GATA4 gene status of the family members shown in FIG. 1.

FIG. 3 are echocardiographs demonstrating the difference between wild-type and GATA4 G296S mutant humans. Representative still frames are shown from transthoracic apical four-chamber view echocardiograms from an unrelated normal child and GATA G296S patient. The arrow indicates dense trabeculation in the body and apex of the right ventricle (RV) of heart with mutation. RA, right atrium; LV, left ventricle; LA, Left atrium.

FIG. 4 is a schematic showing the CRISPR/Cas9 method for correcting a point mutation in iPS cells harboring a point mutation in GATA4. The strategy uses dual nickases, guide RNAs (gRNAs) and a donor DNA cassette,

FIG. 5 are sequencing chromatograms that demonstrated the correction of the point mutation in the iPS cells using the methods as illustrated in FIG. 4.

FIG. 6 is a set of pictures showing the morphologies of the the cell lines following CRISPR editing.

FIG. 7 is a series of karyotypes of the eight established iPS cell lines of the donors described in FIGS. 1 and 2.

FIG. 8 shows expression of select transcripts in the eight established iPS cell lines of the donors described in FIGS. 1 and 2.

FIG. 9 is series of photos showing the staining for pluripotency markers in the eight established iPS cell lines of the donors described in FIGS. 1 and 2.

FIG. 10 is a series of photos showing the ability of the eight established iPS cell lines of the donors described in FIGS. 1 and 2 to differentiate into tissues of all three germ lines in vitro and in vivo.

FIG. 11 is an RNA-seq plot for the established iPS cell lines of the donors described in FIGS. 1 and 2.

FIG. 12 is a series of photos showing the expression of cardiomyocyte markers following the cell differentiation process for generating cardiomyocytes from iPS cells.

FIG. 13 shows RNA-seq analysis, GO analysis and signaling pathway analysis of the iPS cells undergoing cardiomyocyte differentiation. Significance is shown as −Log 10 Bonferroni p-value after multiple hypothesis correction.

FIG. 14 is a series of graphs showing stage-specific gene expression signatures for selected genes representing mesoderm, cardiac progenitor cells (CPCs) and cardiomyocytes.

FIG. 15 is a series of two photos showing the iPS cardiomyocytes displayed gene expressions similar to human cardiomyocytes, with expression of high levels of sarcomeric and myofibril markers.

FIG. 16 illustrates the membrane electrophysiology and ability of the iPS cardiomyocytes to spontaneously contract.

FIG. 17 are graphs showing calcium flux measurements of hiPS cell-derived cardiomyocytes and the expected response to isoproterenol (a β-andrenergic agonist) followed by carbachol (a cholinergic agonist).

FIG. 18 is an electron micrograph of representative iPS-derived cardiomyocytes showing mitochondria, Z-lines and sarcomeres.

FIG. 19 shows FACS analysis of cTnT⁺ cardiomyocytes and differentiation from representative WT and G296S cells after lactate purifications.

FIG. 20 shows cardiomyocytes patterned as arrays of single cells (top) and immunostained for α-actinin or F-actin in physiological (bottom) substrate stiffness (10 kPa) and surface area (2000 μm²). The cells show mature sarcomoric organizations.

FIG. 21 is a set of graphs showing contractile measurements on micro-patterns. The percentage of single-cardiomyocytes responding accurately to 1 Hz electrical pacing in WT and G296S cells is shown on the left. Traction force microscopy measurements of force production as a function of cell displacement of all cardiomyocytes responding accurately to ⋅1 Hz pacing is shown on the right. All measurements were done in triplicate with cardiomyocytes generated independently from 2 patient lines.

FIG. 22 is a graph showing decreased contraction time in WT and G296S cardiomyocytes.

FIG. 23 is a set of graphs showing action potential measurements of WT and G296S cardiomyocytes. Overshoot potential (OSP) is the highest membrane potential reached; dV/dtₘₐₓ, is the maximum upstroke velocity; APD90 is the duration of action potential at 90% repolarization. Data shown are mean±SEM from 2 WT and 2 G296S cell lines. * denotes p<0.05 (Mann-Whitney test).

FIG. 24 is a graph showing calcium flux measurement on microclusters, F/F₀ (Max), peak amplitude relative to baseline fluorescence between action potentials. Data shown are mean±SEM from 2 WT and 2 G296S cell lines. * denotes p<0.05 (Mann-Whitney test).

FIG. 25 is a graph showing mitochondria staining intensity of single cardiomyocyte micropatterns (top). Mitotracker red intensity relative to cell area was quantified (bottom). Data shown are mean:±SEM from 2 G296S cell lines.**, p<0.005 (t test).

FIG. 26 is a graph showing seahorse measurements of glycolytic functions. lsogenic cardiomyocyte data are mean±SEM. **, p<0.005, ***, p<0.0005 (t test).

FIG. 27 is a graph showing de novo mtDNA mutations following genome sequencing of mtDNA from WT and G296S cells.

FIG. 28 is heat map showing hierarchical clustering of Spearman correlation scores for all differentiation time course samples based on RNA-seq profiles. hES, H7, hiPS, WT1, WT_MES, WT1. Dark grey, GATA4 mutants; Score of 1 (light grey) denotes perfect correlation.

FIG. 29 is a human fetal tissue prediction matrix for all differentiation time course samples based on RNA-Seq profiles. Dark grey, GATA4 mutants, Score of 1 (light grey) denotes highest similarity.

FIG. 30 is a heat map showing hierarchical clustering of 2,228 genes differentially expressed at any time point. Dark grey and light grey represent decreased and increased expressions (Log₂ FC) respectively.

FIG. 31 is a set of Venn diagrams showing downregulation (left) and upregulation (right) of differentially expressed genes in CPCs, D15-cardiomyocytes, and D32-cardiomyocytes.

FIG. 32 is a bar graph showing GO analyses (BioPro/Disease/Pathway) of down- and up-regulated genes during the differentiation from CPCs to mature cardiomyocytes. Significance is shown as −Log 10 Bonferroni p-value after multiple hypothesis correction.

FIG. 33 is a heat map showing hierarchical clustering of differentially expressed genes during the differentiation from CPCs to mature cardiomyocytes. Values are row scaled to show relative expression. Dark grey and light grey are high and low levels respectively. Representative down- and up-regulated genes are listed.

FIG. 34 shows the Network2Canves analyses indicating genes enriched for GATA-factor binding, developmentally regulated by p300 and PRC2 complex, and important for cardiovascular development and function.

FIG. 35 is a heat map showing hierarchical clustering of differentially expressed genes in CPCs. Values are row scaled to show relative expression. Dark grey and light grey are high and low levels respectively. Representative down- and up-regulated genes are listed.

FIG. 36 is a bar graph showing GO analyses (BioPro/Disease/Pathway) of down- and up-regulated genes in CPCs. Significance is shown as −Log 10 Bonferroni p-value after multiple hypothesis correction.

FIG. 37 is a scale-free network of 100 nodes with an average 3.1 neighbors and path length of 4.8. Nodes are genes that are down-regulated in G296S CPCs during cardiomyocyte differentiation.

FIG. 38 is a heat map showing hierarchical clustering of differentially expressed genes in D15-cardiomyocytes. Values are row scaled to show relative expression. Dark grey and light grey are high and low levels respectively. Representative down- and up-regulated genes are listed.

FIG. 39 is a bar graph showing GO analyses (BioPro/Disease/Pathway) of down- and up-regulated genes D15-cardiomyocytes. Significance is shown as −Log 10 Bonferroni p-value after multiple hypothesis correction.

FIG. 40 is a heat map showing hierarchical clustering of differentially expressed genes in D32 cardiomyocytes. Values are row scaled to show relative expression. Dark grey and light grey are high and low levels respectively. Representative down- and up-regulated genes are listed.

FIG. 41 is a bar graph showing GO analyses (BioPro/Disease/Pathway) of down- and up-regulated genes D32-cardiomyocytes. Significance is shown as ⋅−Log 10 Bonferronl p-value after multiple hypothesis correction.

FIG. 42 is a graph of Gene Set Enrichment Analyses (GSEA) showing a reduction in cellular respiration genes in WT versus G296S cardiomyocytes.

FIG. 43 is a heat map showing hierarchical clustering of differentially expressed genes in smooth muscle,

FIG. 44 shows GSEA analyses of genesets for cardiac (top) and endothelial/endocardial (bottom) development. NES, normalized enrichment score. FDR, false discovery rate. Positive NES means higher expression in iWT cells. Negative NES means higher expression in G296S cells.

FIG. 45 are IGV browser tracks at chr14:23693015-24168059 showing normalized ATAC-seq signal from WT (black) and G296S (dark grey) cells matches normalized signal from ENCODE-OHS (light grey regions).

FIG. 46 is a heat map of normalized read counts from ENCODE-DHSs; H3K4me3, H3K27me3 (D5CPC) (Stergachis et al., 2013) ±1 kb around the center of ATAC-seq peaks identified in iWT CPCs. White and grey are low and high signal intensity, respectively.

FIG. 47 are pie-charts showing gene-body, upstream, and downstream distribution (top) and coding and non-coding gene distribution (bottom) of 14532 iWT ATAC-seq peaks.

FIG. 48 are IGV browser tracks at TBX5(top) and SOX17 (bottom) loci show decreased and increased (light grey regions) ATAC-seq signal between WT (black) and G296S (dark grey). Scales represent reads/million/25 bp.

FIG. 49 are graphs showing metagenes plots of iWT (black) and G296S (dark grey) normalized ATAC-seq signal ±5 kb around the TSS of genesets for cardiac (top) and endothelial (bottom) development.

FIG. 50 shows known consensus enriched in ATAC-seq peaks up-regulated in G296S CPC.

FIG. 51 is a set of two bar graphs showing GO analyses (BioPro/Disease/Pathway) of down-regulated (top) and up-regulated (bottom) ATAC-seq peaks after generic peaks were filtered out. Significance is shown as −Log 10 Bonferroni p-value after multiple hypothesis correction.

FIG. 52 is a set of four bar graphs showing FPKM values of select, differentially expressed NOTCH (top) and NFAT (bottom) target genes in iWT cells (black) and G296S cells (grey). Data are mean±SD. *, FDR<0.05.

FIG. 53 shows IGV browser tracks of ChIP-seq signal for GATA4, TBX5; H3K27ac, H3K4me3, H3K36me13, H3K27me3 at known target loci (NPPA, NPPB) in WT cardiomyocytes. Grey boxes, significant peaks identified by MACS2. Scales represent reads/million/25 bp.

FIG. 54 shows IGV browser tracks of ChIP-seq signal for GATA4, TBX5; H3K27ac, H3K4me3, H3K36me13, H3K27me3 at additional target loci (TNNT2, TNNT1) in WT cardiomyocytes. Grey boxes, significant peaks identified by MACS2. Scales represent reads/million/25 bp.

GATA4 and TBX5 ChIP-seq signals were also positively correlated to gene expression levels (FIG. 55). Overall, GATA4, TBX5 and H3K27ac shared the strongest (FIG. 56), with nearly half of GATA4 sites being co-bound by TBX5 (FIGS. 56, 57). 2428 sites co-bound by human G4T5 had higher ChIP-seq signals than sites bound by GATA4 or TBX5 alone (FIG. 58). Co-bound sites mostly mapped to intronic (48%) and intergenic (35%) enhancer sites of genes for myofibril assembly, cardiac muscle development and contraction, CHD and cardiomyopathy (FIG. 59, 60).

FIG. 55 is a metagenes plot of normalized ChIP-seq signal for GATA4, TBX5, H3K4me3, H3K27ac H3K36me3 and H3K27me3 for overlap in genome occupancy in WT cardiomyocytes.

FIG. 56 is a heat map of normalized read counts from ENCODE-DHSs; GATA4, TBX5, H3K27ac H3K36me3 and H3K27me3 at 2428 G4T5 co-bound sites (±0.5 kb) identified in WT cardiomyocytes. White and grey are low and high signal intensity, respectively.

FIG. 57 is a Venn diagram showing the GATA4 sites co-bound by TBX5.

FIG. 58 is a set of graphs for normalized GATA4 (left) or TBX5 (right) signal at sites that are G4T5 co-bound versus single transcription factor bound. Boxplot and whiskers show mean, 25^{th} and 75^{th} percentile followed by 5^{th} and 95^{th} percentile. ****,p<0.00005, (Kolmogorov-Smirnov test).

FIG. 59 illustrates the intronic (48%) and intergenic (35%) co-bound sites enhancer sites of genes for GATA4 and TBX5.

FIG. 60 is a bar graph showing GO analyses (BioPro/Disease/Pathway) of down- and up-regulated genes in myofibril assembly, cardiac muscle development and contraction, CHD, cardiomyopathy, etc. Significance is shown as −Log 10 Bonferroni p-value after multiple hypothesis correction.

FIG. 61 are consensus motifs enriched in 2428 G4T5 co-bound sites in WT cardiomyocytes.

FIG. 62 is a Venn diagram showing the co-bound enhancer sites of genes for GATA4 and TBX5 in G296 cardiomyocytes.

FIG. 63 is a metagenes plot of normalized ChIP-seq signal for GATA4, TBX5, H3K27ac H3K36me3 and H3K27me3 at 2428 G4T5 co-bound sites (±0.5 kb) identified in G296S cardiomyocytes.

FIG. 64 is Venn diagram (top) showing changes in GATA4, TBX5 or G4T5 bound sites between WT cells and G296S cells, Number of sites lost in WT (L), gained in G296S (E) and unchanged (U) are shown. Legend for metagenes of relative (G296S/WT) ChIP-seq occupancy ±3 kb around sites that are L, U or E. Bottom, relative changes in GATA4, TBX5, and H3K27ac occupancy at L, U or E sites.

FIG. 65 is a set of graphs showing FPKM values of GATA4, TBX5 and K27ac mapped ±20 kb of G4T5^{L} sites, G4T5^{U} sites and G4T5^{E} sites during iWT (black) and G296S (grey) cardiac differentiation. Boxplot and whiskers show mean, 25^{th} and 75^{th} percentile followed by 5^{th} and 95^{th} percentile. *,p<0.05, **,p<0.005, **″″,p<0.0005, (Wilcoxon signed-rank test).

FIG. 66 shows consensus motifs for in G4T5E sites and G4T5E sites in G296S cardiomyocytes.

FIG. 67 is a series of Venn diagrams showing differential expression of G4T5 co-bound genes in D15 and D32 cardiomyocytes.

FIG. 68 is a graph showing down-regulation of expression levels of genes with decreased GATA4 and TBX5 binding in G296S cardiomyocytes.

FIG. 69 is a set of graphs showing the ChIP-seq signal for H3K4me3 and H3K27me3 in AT (Black line) versus G296S (grey line) cardiomyocytes.

FIG. 70 is a bar graph showing GO analyses (BioPro/Disease/Pathway) of 82 up-regulated endothelial genes in G296S cardiomyocytes. Significance shown as −Log 10 Bonferroni p-value after multiple hypothesis correction.

FIG. 71 is a graph showing the distribution of MED1 ChIP-seq signal across 5,040 putative enhancers in WT cardiomyocytes. 213 SEs show exceptionally high MED⋅1 binding. Representative genes within 20 kb of the 213 SEs are labeled.

IG. 72 shows IGV browser tracks of ChIP-seq signal for GATA4, TBX5, MED1, H3K27ac, at MYH6 and MYH7 loci showing a 47 kb SE element. A 1.3 kb typical enhancer (TE) at STAU2 is shown for comparison. Scales represent reads/million/25 bp.

FIG. 73 is a set of shows IGV browser tracks of ChIP-seq signal for GATA4, TBX5; MED1 and H3K27ac at known target loci in G296S cardiomyocytes. Scales represent reads/million/25 bp.

FIG. 74 is a graph demonstrating a positive correlation between the MED1 ChIP-seq signal and gene expression levels in G296S cardiomyocytes.

FIG. 75 is a set of graphs showing enhancer length (left) and nearest (20 kb) gene expression (right) of TE and SE. Boxplot and whiskers show mean, 25^{th} and 75^{th} percentile followed by 5^{th} and 95^{th} percentile. ****, p<0.00005 (t test).

FIG. 76 is a graph illustrating enhanced binding of MED1, GATA4 and TBX5 binding in SE elements as demonstrated by normalized ChIP-seq signal.

FIG. 77 shows known consensus motifs enriched at constituent enhancers within SE elements in WT cardiomyocytes.

FIG. 78 is a bar graph showing GO analyses (BioPro/Disease/Pathway) of 213 SE elements. Significance shown as −Log 10 Bonferroni p-value after multiple hypothesis correction.

FIG. 79 is a graph showing the distribution of MED2 ChIP-seq signal across all 5,040 enhancers in G296S cardiomyocytes. 172 SE show exceptionally high MED1 binding. Representative genes within 20 kb of the 172 SE are labeled.

FIG. 80 is a Venn diagram (top) showing changes in MED-1-bound SE elements between WT (black circle) and G296S (grey). Number of sites lost in WT (L), gained in G296S (E) or unchanged (U) are shown.

FIG. 81 is a metagenes plot of normalized GATA4 and TBX5 ChIP-seq signal within SE that are L, U or E In WT (black line) and G296S (grey line) cardiomyocytes.

FIG. 82 is a series of graphs showing GATA4, TBX5 and K27ac binding in the SEL SEU and SEE elements in WT and G296S cardiomyocytes, as demonstrated by ChIP-seq signal

FIG. 83 shows select genes within 20 kb of the SE elements that are L, U or E in G296S cardiomyocytes.

FIG. 84 is a graph showing the FPKM values of genes mapped ±20 kb around SE during iWT (black) and G296S (grey) cardiac differentiations. Boxplot and whiskers show mean, 25^{th} and 75^{th} percentile followed by 5^{th} and 95^{th} percentile. *p<0.05, *** p<0.0005, ****, p<0.00005 (Wilcoxon signed-rank test).

FIG. 85 is a bar graph showing the % of genes with SE elements in G296S cardiomyocytes.

FIG. 86 is a bar graph showing the decrease in contractility following knockdown of SE elements in cardiomyocytes.

FIG. 87 is a bar graph showing the abnormalities in calcium flux following knockdown of SE elements in cardiomyocytes.

FIG. 88 is a bar graph showing the decrease in mitochondria mass following knockdown of SE elements in cardiomyocytes.

FIG. 89 illustrates a collapse of the core cardiac transcriptional network due to depletion of MALAT1 and KLF9.

FIG. 90 is a scale-free network of 716 nodes connected by 2,353 edges with an average 6.6 neighbors and path length of 4.3. Nodes are genes that are differentially expressed or G4T5 co-bound or have MED-1 SE elements. Edges are physical or functional interactions between nodes as extracted from STRING. Hubs are grouped into 5 sub-networks.

FIG. 91 is a sub-network plot of extracted top-20 hubs named by gene symbol. Number of edges from entire GRN is shown beside each node. Diamond, square, and circle represent genes that gained or lost G4T5 binding or were unchanged, respectively. Bolded border represents genes with SE elements.

FIG. 92 is a bar chart showing gene ontology analysis for expression in the GATA4-TBX5 controlled gene regulatory network

FIG. 93 is a set of graphs showing force generation of engineered cardiomyocytes treated with a PI3K inhibitor (left) or PI3K activator (right). Relative change in force generation between iWT (black) and G296S (grey) cardiomyocytes after inhibition (circle) or activation (triangle) of P13K signaling. Traction force microscopy (TFM) measurements of CMs responding accurately to 1 Hz pacing. Data are mean±sem, *,p<0.05, **,p<0.005, ***p<0.0005 (Mann-Whitney test).

FIG. 94 is a schematic illustrating the postulated protein-protein interaction in wild-type (top) and G296S mutant (bottom) cardiomyocytes. Beat rate measurements between iWT (black) and G296S (grey) cardiomyocytes after inhibition (circle) or activation (triangle) of P13K signaling. TFM measurements of cardiomyocytes responding accurately to 1 Hz pacing. Data are mean±sem, *,p<0.05, ** p<0.005, ***p⋅<0.0005 (Mann-Whitney test).

FIG. 95 is a set of graphs showing the effect on beat rate of wild-type and G296S engineered cardiomyocytes treated with a PI3K inhibitor (left) or PI3K activator (right). Top, cardiac gene loci in WT are open and permissive to G4T5 binding at MED1-bound SE elements, which activates transcription; G4T5 also binds and prevents aberrant open chromatin and transcription at endothelial genes. Bottom, transcriptional and epigenetic consequences of GATA4 G296S.

FIG. 96 is a TSNE plot of single cell RNA-sequencing data, with each cell represented by a single dot, clustered into distinct groups. The panel on the left represents cells from normal human iPS cells differentiated toward the cardiomyocyte lineage for 7 days, while the panel on the right is the same with human cells carrying a GATA4 mutation. Differences can be observed at the single cell level with the most obvious being a cluster absent in the mutant cells.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “antibody” is intended to include any polypeptide chain-containing molecular structure with a specific shape that fits to and recognizes an epitope, where one or more non-covalent binding interactions stabilize the complex between the molecular structure and the epitope. As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Where bispecific antibodies are to be used, these may be conventional bispecific antibodies, which can be manufactured in a variety of ways (Holliger and Winter, Curr Opin Biotechnol. 1993 August; 4(4):446-9), e.g., prepared chemically or from hybrid hybridomas, or may be any of the bispecific antibody fragments mentioned above. It may be preferable to use scFv dimers or diabodies rather than whole antibodies. Diabodies and scFv dimers can be constructed without an Fc region, using only variable domains, potentially reducing the effects of anti-idiotypic reaction. Other forms of bispecific antibodies include the single chain “Janusins” described in Traunecker A et al., EMBO J. 1991 December; 10(12):3655-9. Such antibodies also include CRAbs, which are chelating antibodies which provide high affinity binding to an antigen, D. Neri, et al. J. Mol. Biol, 246, 367-373, and dual-variable domain antibodies as described in Wu C et al., Nat Biotechnol. 2007 November; 25(11):1290-7. Epub 2007 Oct. 14.

A “candidate agent” as used herein refers to any agent that is a candidate to treat a disease or symptom thereof. Examples of candidate agents that can be used in the methods of the disclosure include, but are not restricted to: small molecules; peptides; proteins (including derivatized or labeled proteins); siRNA molecules; CRISPR-based therapeutic agents; antibodies or fragments thereof; aptamers; carbohydrates and/or other non-protein binding moieties; derivatives and fragments of naturally-occurring binding partners; and peptidomimetics.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

The term “pharmaceutically acceptable carrier” as used herein is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. The use of such media and agents is well known in the art. Except insofar as any conventional media or agent is incompatible with the agents provided herein, use thereof in the composition is contemplated.

The terms “peptide,” “polypeptide,” and “protein” are used interchangeably herein, and refer to a polymeric form of amino acids of any length, which can include coded and non-coded amino acids, chemically or biochemically modified, labeled or derivatized amino acids, and polypeptides having modified peptide backbones.

The term “peptidomimetic” as used herein refers to a protein-like chain designed to mimic a peptide. They typically arise from modification of an existing peptide in order to alter the molecule's properties. For example, they may arise from modifications to change a molecule's stability, biological activity, or bioavailability.

The term “small molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in an animal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### DETAILED DESCRIPTION OF THE INVENTION

The practice of the methods and compositions described herein may employ, unless otherwise indicated, conventional techniques of pharmaceutical chemistry, drug formulation techniques, dosage regimes, and biochemistry, all of which are within the skill of those who practice in the art. Such conventional techniques include the use of combinations of therapeutic regimes including but not limited to the methods described herein; technologies for formulations of adjunct therapies used in combination with known, conventional therapies and/or new therapies for the treatment of cardiac diseases and disorders, delivery methods that are useful for the compositions of the invention, and the like.

Specific illustrations of suitable techniques can be had by reference to the examples herein. However, other equivalent conventional procedures can, of course, also be used. Such conventional techniques and descriptions can be found in standard laboratory manuals such as The practice of the present invention employs, unless otherwise indicated, conventional techniques of immunology, biochemistry, chemistry, molecular biology, microbiology, cell biology, genomics and recombinant DNA, which are within the skill of the art. See Green and Sambrook, (MOLECULAR CLONING: A LABORATORY MANUAL. 4th, ed., Cold Spring Harbor Laboratory Press, Cold Spring Harbor, N.Y., 2014); CURRENT PROTOCOLS IN MOLECULAR BIOLOGY (F. M. Ausubel, et al. eds., (2017)); Short Protocols in Molecular Biology, (Ausubel et al., 1999)); the series METHODS IN ENZYMOLOGY (Academic Press, Inc.): PCR 2: A PRACTICAL APPROACH (M. J. MacPherson, B. D. Hames and G. R. Taylor eds. (1995)), ANTIBODIES, A LABORATORY MANUAL, SECOND EDITION (Harlow and Lane, eds. (2014) and CULTURE OF ANIMAL CELLS A MANUAL OF BASIC TECHNIQUE, 7TH EDITION (R. I. Freshney, ed. (2016)).

For further elaboration of general techniques useful in the practice of this invention, the practitioner can refer to standard textbooks and reviews in cell biology, tissue culture, embryology, and cardiophysiology. With respect to tissue culture and embryonic stem cells, the reader may wish to refer to EMBRYONIC STEM CELLS: A PRACTICAL APPROACH (Notarianni and Evans, ed., IRL Press Ltd. 2006); ANIMAL CELL CULTURE AND TECHNOLOGY (THE BASICS), M Butler (2004) second edition; HUMAN EMBRYONIC STEM CELL PROTOCOLS (METHODS IN MOLECULAR BIOLOGY) K Turksen (2015); HUMAN STEM CELL MANUAL, SECOND EDITION: A LABORATORY GUIDE S Peterson and J F. Loring (2012); and HUMAN EMBRYONIC STEM CELLS (ADVANCED METHODS (BIOS)), R. Pedersen and Jon Odorico (2007). With respect to the culture of heart cells, standard references include The Heart Cell in Culture (A. Pinson ed., CRC Press 1987), Isolated Adult Engineered cardiomyocytes (Vols. I & II, Piper & Isenberg eds, CRC Press 1989), Heart Development (Harvey & Rosenthal, Academic Press 1998).

Note that as used herein and in the appended claims, the singular forms “a,” “an,” and “the” include plural referents unless the context clearly dictates otherwise. Thus, for example, reference to “a cell” refers to one or more cells with various pluripotency and expression patterns, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### The Invention in General

The present disclosure provides methods of modulating aberrant transcription factor regulation, and in particular aberrant regulation of the PI3K pathway, e.g. GATA and/or TBX5.

GATA4 is a transcription factor highly expressed throughout development in myocardial, endocardial and endodermal cells (Cirillo et al., 2002; Heikinheimo et al., 1994; et Zeisberg al., 2005). Heterozygous mutations in GATA4, a cardiogenic transcription factor, cause congenital heart defects and cardiomyopathy through unknown mechanisms. Gata4 deletion in mice causes malformations in extraembryonic and foregut endoderm (Kuo et al., 1997; Molkentin et al., 1997), and Gata4 is essential in regulating engineered cardiomyocyte (cardiomyocyte) proliferation and ventricular septal development (Rojas et al., 2008; Zeisberg et al., 2005). Conditionally deleted Gata4 mice undergo cardiac decompensation from cardiomyocyte apoptosis (Oka et al., 2006) and GATA4^{+/-} mice have cardiac hypoplasia and reduced hypertrophic response to pressure overload (Bisping et al., 2006). Thus, GATA4 is essential for cardiac development and homeostasis in mice.

Super enhancers (SEs), which are clusters of putative enhancers densely occupied by a mediator complex and cell fate-determining transcription factors, have been implicated as central regulators of cell identity in development and disease. SEs differ from typical-enhancers (TEs) in size, transcription factor motif density and transcriptional activation capacity (Whyte et al., 2013). The dense clustering of transcription factor-bound enhancer sites and coactivator enrichment render SEs more sensitive to alterations in molarity of TF complexes (Adam et al., 2015). SE gene regulation is implicated in B-cell genome stability (Meng et al., 2014), T cell specification (Vahedi et al., 2015), hair follicle stem cell plasticity (Adam et al., 2015) and acute myeloid leukemia (Pelish et al., 2015). However, the present invention is the first demonstration that SE gene regulation by cardiac transcription factors has a significant role in human cardiac development or disease.

Heterozygous GATA4 and TBX5 mutations cause familial congenital heart defects with overlapping phonotypes, and these factors have been shown co-immunoprecipitate upon overexpression (Besson et al., 1997; Garg et al., 2003; Li et al., 1997). A disease-causing GATA4 G2968 missense mutation affecting a residue at the base of the second zinc finger impaired in vitro Interaction between GATA4 and TBX5 was previously reported (Garg et at, 2003). Mice with compound heterozygous GATA4 and TBX5 mutations developed atrioventricular septal defects (AVSD), providing genetic evidence for their interaction (Maitra et al., 2009), GATA4 and TBX5 are mutated in sporadic CHD in approximately 5% of cases (Rajagopal et al., 2007: Wang et al., 2013), and more recently have been associated with cardiomyopathies (Li et al., 2013; Zhao et al., 2014).

A multi-disciplinary approach with patient derived induced pluripotent stem (iPS) cells was used to better elucidate the GATA4 and TBX5 regulatory mechanisms in human cardiac development and function. The heterozygous GATA4-G296S mutation impairs normal expression of the cardiac gene program and inappropriately upregulated genes of alternative fates, particularly those of the endothelial lineage. These were accompanied by a disruption to GATA4 and TBX5 binding at SE elements associated with genes required for heart development and muscle contraction, and failed chromatin closure at endothelial loci. Computational prediction identified P13K signaling as “hubs” in the GATA4-TBX5 controlled gene regulatory network, and functional tests validated an abnormal P13K episome In GATA4 mutants.

Computational prediction identified P13K signaling as a hub In a GATA4-TBX5 controlled network, supported by functional tests. These results reveal how human disease-causing mutations disrupt transcriptional codes, leading to aberrant chromatin states and cellular dysfunction. GATA4 co-occupied cardiac enhancers with TBX5, another cardiogenic transcription factor. GATA4 G296S mutation disrupted recruitment of TBX5 to human cardiac super enhancers, resulting in diminished cardiac gene expression. Conversely, GATA4-G296S mutation led to GATA4 and TBX5 mislocalization to non-cardiac genes, and enhanced open chromatin states at these loci, particularly at endothelial/endocardial promoters, Correspondingly, GATA4 mutants failed to silence endothelial/endocardial gene expression as part of a broader dysregulation of cell identity.

Candidate agents for therapeutic use in the methods of the invention include any molecule that selectively modulates a target molecule associated with cardiac development and/or function. For the purposes of the present invention, the candidate agent may be a compound that facilitates binding of a molecule with a member of a protein signaling complex, or a compound that interferes with binding of a molecule to its target.

The present disclosure provides physiologically relevant cell culture models and method of use. The disclosure provides mammalian engineered cardiomyocytes generated ex vivo and comprising mutations in the GATA gene, TBX5 gene, and/or the PI3K pathway that are associated with mammalian disease, and in particular with cardiomyopathy in humans.

The present disclosure also provides physiologically relevant cell culture models and method of use. The disclosure provides mammalian engineered cardiomyocytes generated ex vivo and comprising mutation in the GATA gene that are associated with mammalian disease, and in particular with cardiomyopathy in humans.

Importantly, the finding that normal GATA4 activity limits P13K signaling in human cardiomyocytes, and in GATA4 mutants display dysregulated P13K signaling, modulation of P13K signaling, e.g., by using an inhibitor such as LY294002 to modulate PI3K activity in cardiac tissue, may ameliorate this dysfunction. Thus PI3K inhibitors may be able to treat various forms of cardiomyopathy, including but not limited to genetic cardiomyopathy. In other aspects, activating the PI3K pathway, e.g., using an insulin receptor substrate (IRS) synthetic peptide, may also be used to modulate cardiac function in cardiac tissue.

Methods are provided for the generation and use of in vitro cell cultures of disease-relevant engineered cardiomyocytes, where the engineered cardiomyocytes are differentiated from induced human pluripotent stem cells (iPS cells) comprising at least one allele encoding a mutation associated with a cardiac disease, as described above. In some embodiments a panel of such engineered cardiomyocytes is provided, where the panel includes two or more different disease-relevant engineered cardiomyocytes. In some embodiments a panel of such engineered cardiomyocytes are provided, where the engineered cardiomyocytes are subjected to a plurality of candidate agents, or a plurality of doses of a candidate agent. Candidate agents include small molecules, i.e. drugs, genetic constructs that increase or decrease expression of an RNA of interest, electrical changes, and the like.

Methods are also provided for determining the activity of a candidate agent on a disease-relevant engineered cardiomyocyte, the method comprising contacting the candidate agent with one or a panel of engineered cardiomyocytes differentiated from induced human pluripotent stem cells (e.g., iPS cells) comprising at least one allele encoding a mutation associated with a cardiac disease; and determining the effect of the agent on morphologic, genetic or functional parameters, including without limitation calcium transient amplitude, intracellular Ca²⁺ level, cell size contractile force production, beating rates, sarcomeric α-actinin distribution, and gene expression profiling.

In screening assays for the small molecules, the effect of adding a candidate agent to cells in culture is tested with a panel of cells and cellular environments, where the cellular environment includes one or more of: electrical stimulation including alterations in ionicity, drug stimulation, and the like, and where panels of cells may vary in genotype, in prior exposure to an environment of interest, in the dose of agent that is provided, etc., where usually at least one control is included, for example a negative control and a positive control. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the altering of the environment is assessed by monitoring multiple output parameters, including morphological, functional and genetic changes.

In the screening assays for genetic agents, polynucleotides are added to one or more of the cells in a panel in order to alter the genetic composition of the cell. The output parameters are monitored to determine whether there is a change in phenotype. In this way, genetic sequences are identified that encode or affect expression of proteins in pathways of interest. The results can be entered into a data processor to provide a screening results dataset. Algorithms are used for the comparison and analysis of screening results obtained under different conditions.

Methods for analysis include calcium imaging, where cells are loaded with an appropriate dye and exposed to calcium in a condition of interest, and imaged, for example with a confocal microscope. Ca²⁺ responses may be quantified, and the time-dependent Ca²⁺ response was then analyzed for irregularities in timing of successive Ca²⁺ transients and for the total Ca²⁺ influx per transient. The total Ca²⁺ released during each transient was determined by integrating the area underneath each wave with respect to the baseline.

Atomic force microscopy (AFM) can be used to measure contractile forces. Beating cells are interrogated by AFM using a cantilever. To measure forces, cells are gently contacted by the cantilever tip, then the cantilever tip remains in the position for intervals while deflection data are collected. Statistics can be calculated for the forces, intervals between beats, and duration of each contraction for each cell.

Cells can also analyzed by microelectrode array (MEA), where beating engineered cardiomyocytes are plated on MEA probes, and the field potential duration (FPD) measured and determined to provide electrophysiological parameters.

Methods of analysis at the single cell level are of particular interest, e.g., as described above: atomic force microscopy, microelectrode array recordings, patch clamping, single cell PCR, calcium imaging, flow cytometry and the like.

Where the disease is dilated cardiomyopathy (DCM), the engineered cardiomyocytes may be stimulated with positive inotropic stress, such as a β-adrenergic agonist before, during or after contacting with the candidate agent. In some embodiments the β-adrenergic agonist is norepinephrine. It is shown herein that DMC engineered cardiomyocytes have an initially positive chronotropic effect in response to positive inotropic stress, that later becomes negative with characteristics of failure such as reduced beating rates, compromised contraction, and significantly more cells with abnormal sarcomeric a-actinin distribution. β-adrenergic blocker treatment and over-expression of sarcoplasmic reticulum Ca²⁺ ATPase (Serca2a) improve the function. DCM engineered cardiomyocytes may also be tested with genetic agents in the pathways including factors promoting cardiogenesis, integrin and cytoskeletal signaling, and ubiquitination pathway. Compared to the control healthy individuals in the same family cohort, DCM engineered cardiomyocytes exhibit decreased calcium transient amplitude, decreased contractility, and abnormal sarcomeric α-actinin distribution.

Where the disease is hypertrophic cardiomyopathy (HCM) the engineered cardiomyocytes may be stimulated with positive inotropic stress, such as a β-adrenergic agonist before, during or after contacting with the candidate agent. Under such conditions, HCM engineered cardiomyocytes display higher hypertrophic responses, which can be reversed by a β-adrenergic blocker. Compared to healthy individuals, HCM engineered cardiomyocytes exhibit increased cell size and up-regulation of HCM related genes, and more irregularity in contractions characterized by immature beats, including a higher frequency of abnormal Ca²⁺ transients, characterized by secondary immature transients. These engineered cardiomyocytes have increased intracellular Ca²⁺ levels, and in some embodiments candidate agents that target calcineurin or other targets associated with calcium affinity.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select candidate therapeutic agents, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs. The quadrature-anomer of deoxyribose may be used, where the base is inverted with respect to the natural quadrature-anomer. The 2′-OH of the ribose sugar may be altered to form 2′-O-methyl or 2′-O-allyl sugars, which provides resistance to degradation without comprising affinity.

Agents are screened for biological activity by adding the agent to at least one and usually a plurality of cells, in one or in a plurality of environmental conditions, e.g. following stimulation with a β-adrenergic agonist, following electric or mechanical stimulation, etc. The change in parameter readout in response to the agent is measured, desirably normalized, and the resulting screening results may then be evaluated by comparison to reference screening results, e.g. with cells having other mutations of interest, normal engineered cardiomyocytes, engineered cardiomyocytes derived from other family members, and the like. The reference screening results may include readouts in the presence and absence of different environmental changes, screening results obtained with other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype. Both single cell and multicell multiplex assays are amenable to use in the disclosures of the adventure.

The quantitation of nucleic acids, especially messenger RNAs, can also be used for the detection of a candidate agent. These can be measured by sequence techniques, such as RNA-seq; hybridization techniques that depend on the sequence of nucleic acid nucleotides, including array-based detection methods and polymerase chain reaction methods. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

The comparison of screening results obtained from a test compound and a reference screening results(s) is accomplished by the use of suitable deduction protocols, AI systems, statistical comparisons, etc. Preferably, the screening results are compared with a database of reference screening results. A database of reference screening results can be compiled. These databases may include reference results from panels that include known agents or combinations of agents, as well as references from the analysis of cells treated under environmental conditions in which single or multiple environmental conditions or parameters are removed or specifically altered. Reference results may also be generated from panels containing cells with genetic constructs that selectively target or modulate specific cellular pathways.

The readout of the assay may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

The present invention is based on the elucidation that the GATA4 G296S mutation, which is known to cause human disease, impairs contractility, calcium handling and metabolic activity in an in vitro cell population. The broad dysregulation of sarcomeric genes and metabolic genes provides a physiological rationale for the observed defects in human cardiomyocytes. It also provides new targets for the treatment of patients with diseases of cardiac function, e.g., cardiomyopathy.

As shown herein, GATA4 is critical for cardiac and endothelial cell gene regulation in CPCs. GATA4's function as a positive driver of cardiogenesis is unambiguous, but its potential as a repressor of endocardial/endothelial cell fate was previously unknown. Sc1/Tal1 promotes the hematopoietic gene program in hemogenic endothelium and prevents mis-specification into the cardiomyogenic fate (Van Handel et al., 2012). The data provided herein shows that a disease-causing mutation of a transcription factor that normally promotes cardiogenesis induces an ectopic endothelial gene program during human cardiomyocyte differentiation. Indeed, TALI was upregulated by 3.5-fold in G296S CPCs, and may contribute to aberrant endothelial gene expression. Furthermore, G4T5 sites were enriched for motifs of key regulators of hemogenic endothelium, FOXO1 and HOXB4, and G4T5 occupancy normally was associated with repression of gene expression at these sites. However, in GATA4 G296S mutants, loci of inappropriately open chromatin were enriched for motifs of endothelial regulators such as FOXO1 and numerous ETS factors, suggesting loss of G4T5 repression at these sites.

The present disclosure demonstrates a combinatorial transcription factor binding code required for activation of the human cardiac gene program, consistent with that observed in mouse cardiomyocytes (Luna-Zurita et al.), and reveals the epigenetic and transcriptional consequences of a GATA4 missense mutation linked to congenital heart malformations and cardiomyopathy. The ATAC-seq analyses of open chromatin signature and genome-wide profiling of GATA4 and TBX5 binding sites provides a detailed catalog of transcription factor-bound cardiac enhancers in humans. This information pinpoints known and unknown transcriptional regulators and long noncoding MAs that may play important roles in human cardiac development and function.

Interestingly, TEs appear to have a different transcription factor binding code than SEs. In GATA4 mutants, TBX5 binding was decreased at SEs, but increased at many TEs. This difference is consistent with cardiac transcription factors operating via a diverse set of rules of engagement at various enhancer sites, dictated by the underlying cis-sequence and/or the local chromatin configuration (Spitz and Furlong, 2012).

The disclosure describes the mechanisms underlying the requirement for human GATA4 in maintaining cardiomyocyte function and repressing endothelial/endocardial gene expression. Indeed, septal formation and atrioventricular septal development were among the top GO categories of genes dysregulated in GATA4 G296S cardiomyocytes.

The following references are cited herein and/or may be useful for the teachings within the disclosure:
Aanhaanen, W. T., et al. (2011) J Clin Invest 121, 534-544.
Adam, R. C., et al. (2015) Nature 521, 366-370.
Ang, Y. S., et al. (2011). Cell 145, 183-197.
Ang, Y. S. et al., (2016) Cell, 167, Issue 7, p 1734-1749
Ashraf, H., et al. (2014) Circ Cardiovasc Genet 7, 423-433.
Barabasi, A. L., and Albert, R. (1999). Science 286, 509-512.
Sasson, C. T., et al. (1997) Nat Genet 15, 30-35
Bisping, E., et al. (2006) Proc Natl Acad Sci USA 103, 14471-14476.
Bisping, E., et al. (2012) J Biol Chem 287, 9827-9834.
Bruneau, B. G. (2008) Nature 451, 943-948.
Bruneau, B. G., et al. (2001) Cell 106, 709-721.
Buenrostro, J. D., et al. (2013) Nat Methods 10, 1213-1218.
Cirillo, L. A., et al., (2002) Mol Cell 9, 279-289.
Clarke, R. L., et al., (2013). Nat Cell Biol 15, 502-510.
Combs, M. D., and Yutzey, K. E. (2009). Circ Res 105, 408-421.
Garg, V., et al. (2003). Nature 424, 443-447.
He, A., et al., (2011). Proc Natl Acad Sci USA 108, 5632-5637.
Heikinheimo, M., et al., Dev Biol 164, 361-373.
Hutchins, l. M., and Schaefer, S. (2012). Exp Clin Cardiel 17, 81-83.
Ieda, M., et al., (2010). Cell 142, 375-386.
Kathiriya, l. S., et al., (2004) J Biol Chem 279, 54937-54943.
Kuo, C. T., et al., (1997). Genes Dev 11, 1048-1060.
Li, Q. Y., et al., (1997) Nat Genet 15, 21-29.
Li, R. G., et al. (2013) Biochem Biophys Res Commun 439, 591-596.
Lian, X., et al., (2012) Proc Natl Acad Sci USA 109, E1848-E1857.
Luna-Zurita, L, et al. Cell, 164, 999-1014.
Maitra, M., et al., (2012). Curr Top Dev Biol 100, 253-277.
McMullen, J. R., et al. (2004). J Biol Chem 279, 4782-4793.
Meng, F. L, Du, Z., et al. (2014). Cell 159, 1538-1548.
Merkle, F. T., and Eggan, K. (2013) Cell Stem Cell 12, 656-668.
Misra, C., et al., (2012) PLoS Genet 8, e1002690.
Molkentin, J. D et al., (1997) Genes Dev 11, 1061-1072.
Oka, T., et al., (2006). Circ Res 98, 837-845.
Okita, K., et al. (2011) et al. (2015) Nature 526, 273-276.
Qian, L, et al., (2012) Nature 485, 593-598.
Rajagopal, S K, et al. (2007). J Mol Cell Cardiol 43, 677-685.
Ribeiro, A. J., et al., (2015) Proc Natl Acad Sci USA 112, 12705-12710.
Rojas, A., (2008) Mol Cell Biol 28, 5420-5431.
Roost, M. S., (2015) Stem Cell Reports 4, 1112-1124.
Shen, B., et al. (2014). Nat Methods 11, 399-402.
Song, K., et al. (2012). Nature 485, 599-604.
Spencer, C. J., et al. (2014) Stem Cell Reports 3, 269-281.
Spitz, F., and Furlong, E. E. (2012). Nat Rev Genet 13, 613-626.
Srivastava, D. (2006). Annu Rev Pathol 1, 199-213.
Stefanovic, et al., (2014). Nat Commun 5, 3680.
Stergachis, A. B., et al. (2013) Cell 154, 888-903.
Stewart, J. B., and Chinnery, P. F. (2015) Nat Rev Genet 16, 530-542.
Tan, C. M., et al., Bioinformatics 29, 1872-1878.
Tohyama, S., et al. (2013) Cell Stem Cell 12, 127-137.
Vahedi, et al. (2015). Nature 520, 558-562.
Van Handel, B., et al. (2012) Cell 150, 590-605.
Wang, E., et al., (2013) Cell 153, 307-319.
Xu, H., et al., (2014). PLoS Comput Biol 10, e1003777.
Zaret, K. S., and Carroll, J. S. (2011) Genes Dev 25, 2227-2241.
Zeisberg, E. M., et al., (2005) J Clin Invest 115, 1522-1531.
Zhao, L., (2014) Int J Mal Med 33, 654-660.

### EXAMPLES

The following examples are put forth so as to provide those of ordinary skill in the art with a complete disclosure and description of how to make and use the present invention, and are not intended to limit the scope of what the inventors regard as their invention, nor are the examples intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1: Generation of Patient-Specific IPS Cells and Functional Engineered Cardiomyocytes

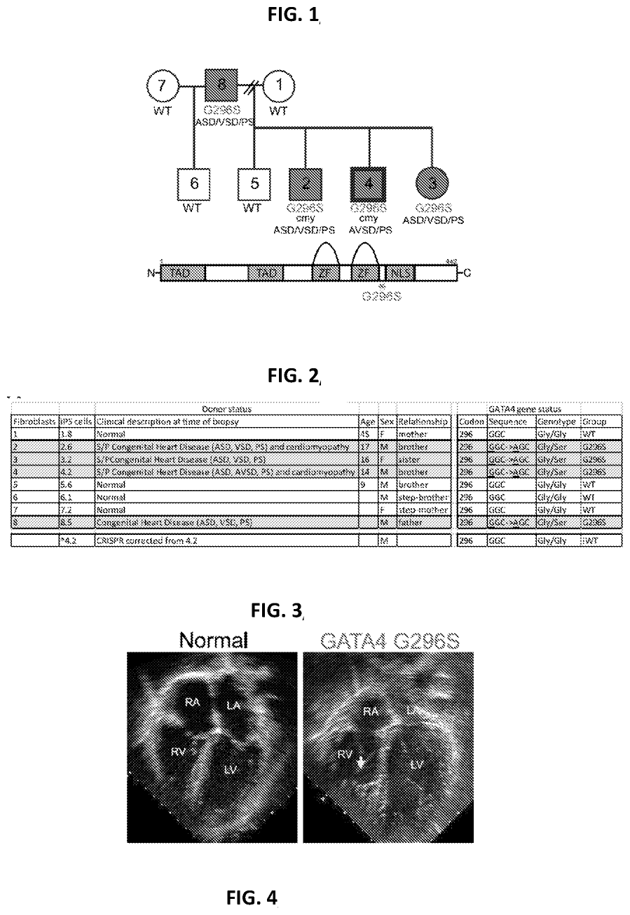

A heterozygous c.886G>A mutation in human GATA4 was linked to 100% penetrant atrial or ventricular septal defects, AVSD and pulmonary valve stenosis (PS) (FIGS. 1 and 2) (Garg et al., 2003). Mutant GATA4 translated into a G296S missense substitution flanking the zinc finger domain, a region involved In DNA-binding and protein-protein interactions (FIG. 1, bottom). Several GATA4 G296S patients were identified who developed a delayed-onset cardiomyopathy in their teenage years. This was characterized by decreased left ventricular systolic function and an unusual echocardiographic appearance of the right ventricle with deep trabeculations and thickening of the papillary muscles in the left ventricle (FIG. 3). The deep trabeculation is typical of a condition known as non-compaction, thought to reflect a failure of ventricular cardiomyocytes to mature (Hutchins and Schaefer, 2012). This clinical observation connected the heterozygous GATA4 mutation to a broad misregulation of cardiomyocyte identity and function via perturbation of key transcriptional complexes.

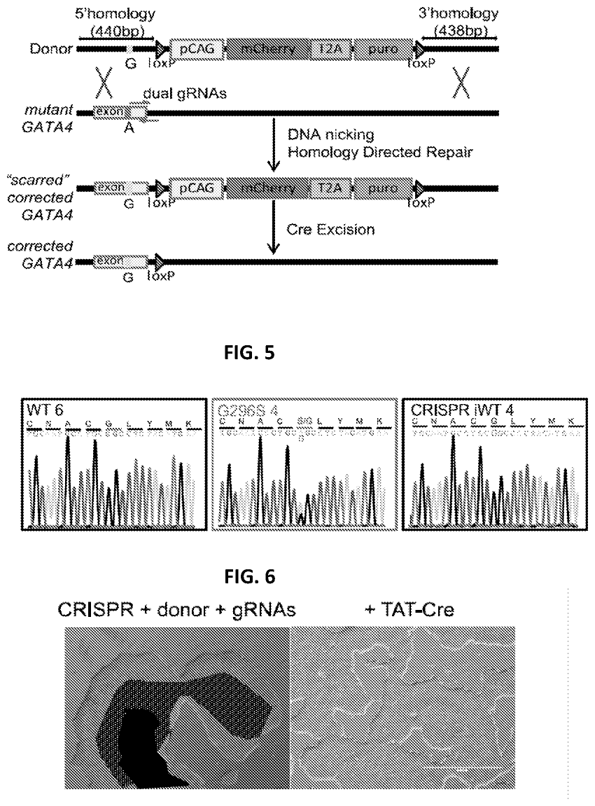

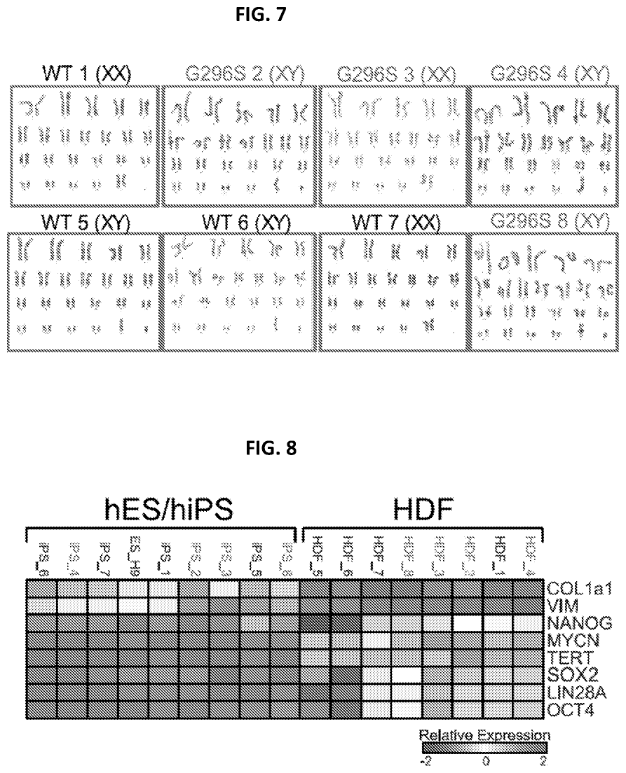

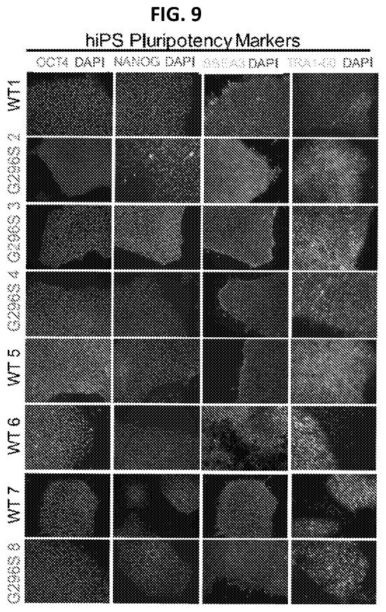

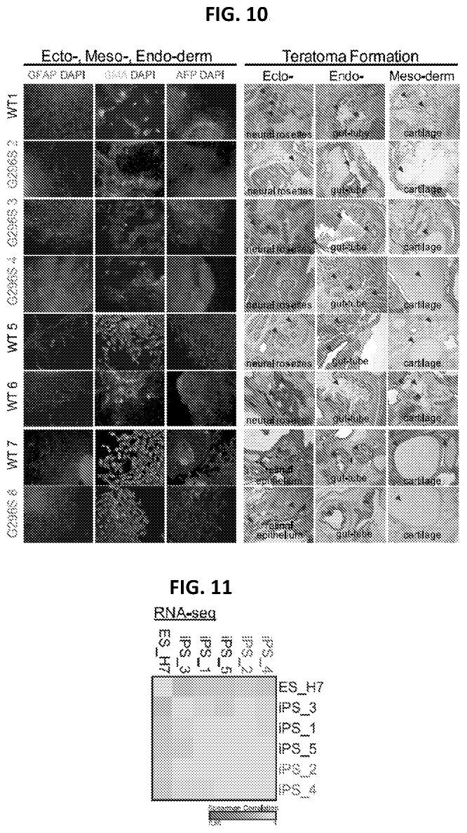

The mechanism by which a dose-sensitive missense mutation causes defects in human cardiac development and function was investigated by reprogrammed dermal fibroblasts obtained from four subjects harboring the GATA4 G296S mutation, and four family members without the mutation, into patient-specific iPS cells (Okita et al., 2011) (FIG. 1). CRISPR/Cas9 nucleases were used to edit the point mutation (A) back to its wildtype sequence (G) in iPS cells of patient 4 to yield isogenic wild type controls (iWT) (FIGS. 4 and 5). All iPS cell lines and CRISPR-edited clones propagated robustly, had ES cell-like morphologies, normal karyotypes, expressed self-renewal markers and had silenced HDF markers (FIG. 6-9). RNA sequencing (RNA-seq) confirmed a genome-wide correlation in gene expression signature between ES and iPS cells (FIG. 10). All iPS lines were differentiated into the three germ layers in vitro and in vivo, demonstrating pluripotency (FIG. 11).

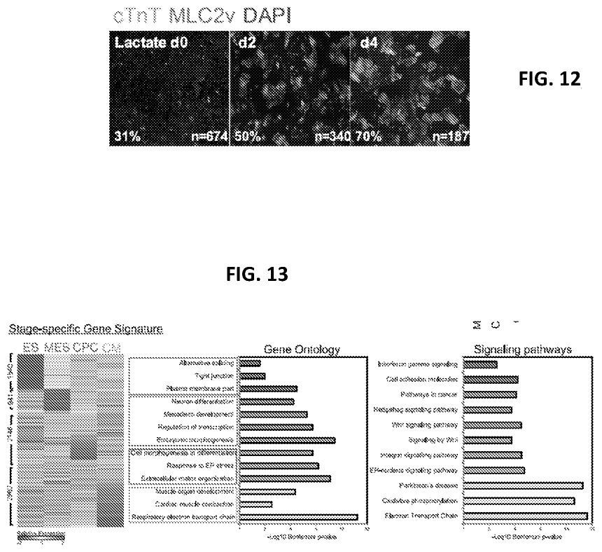

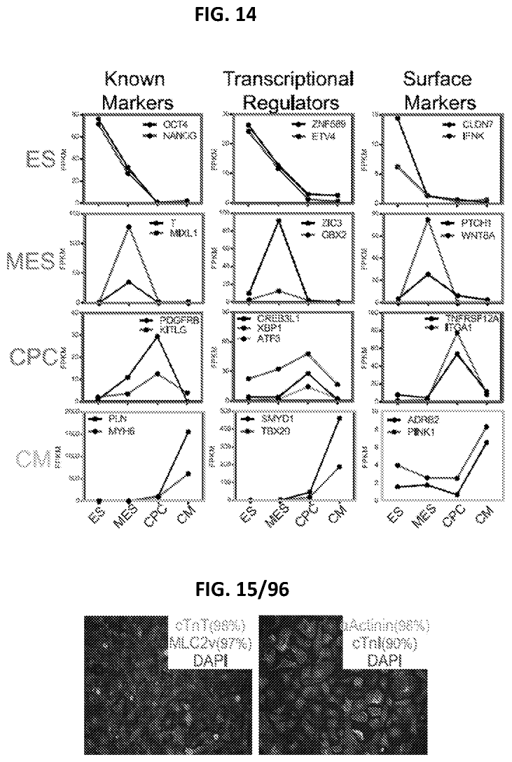

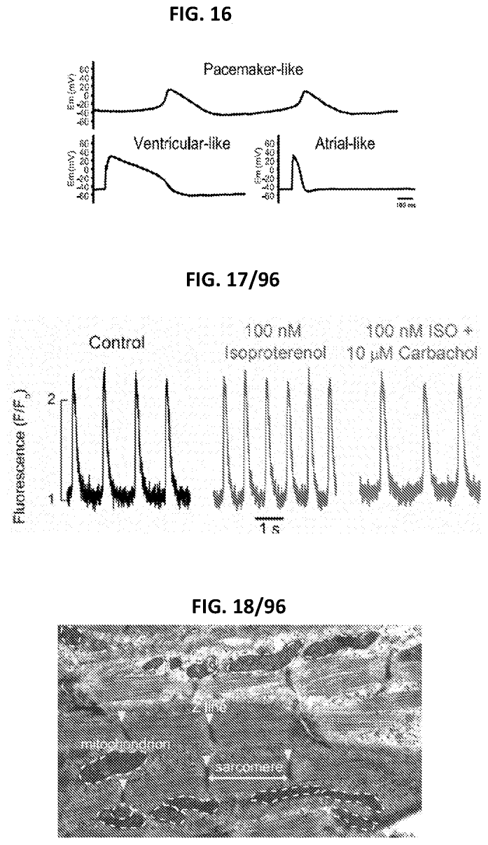

A step-wise differentiation protocol was used to generate purified cardiomyocytes from the above-described iPS cell lines (FIG. 12) (Lian et al, 2012; Tohyama et al.,2013). RNA-seq at various time points showed stage-specific gene signatures representing mesoderm, cardiac progenitor cells (CPCs) and cardiomyocytes with expected gene ontologies (GO) enriched at each stage (FIGS. 13 and 14). The iPS cardiomyocytes spontaneously contracted, expressed high levels of sarcomeric and myofibril markers, and had membrane electrophysiology and gene expressions similar to human cardiomyocytes; 30% were binucleated (FIGS. 15 and 16). Calcium flux measurements showed proper drug responses while electron microscopy indicated abundant mitochondria with defined Z lines and sarcomeres (FIGS. 17 and 18). The protocol thus generated functional human cardiomyocytes suitable for deeper interrogation of cell identity and function.

### Example 2: Impaired Contractility, Calcium Handling, Metabolic Activity In GATA4 Mutant Engineered Cardiomyocytes

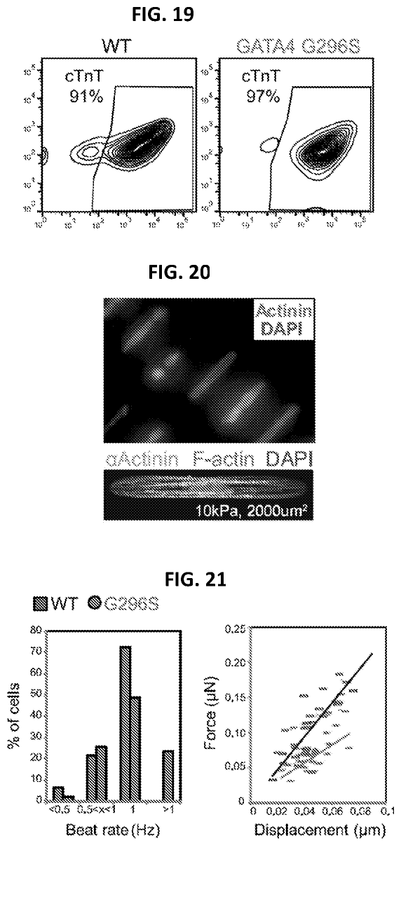

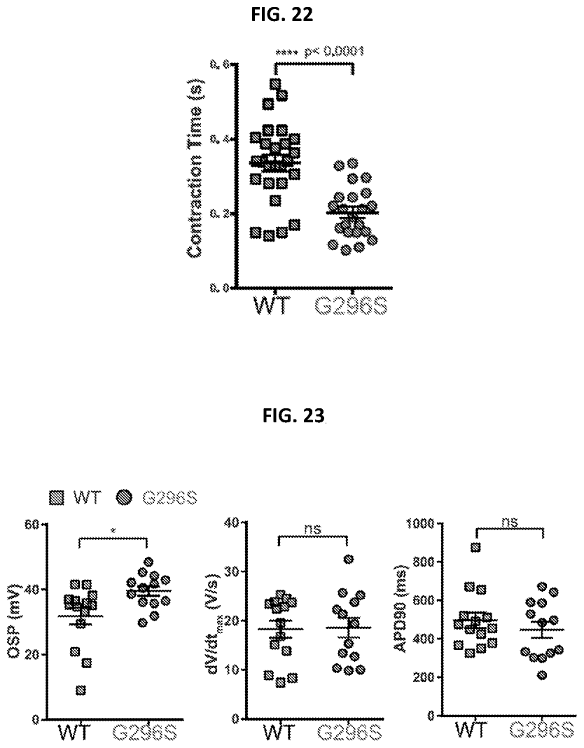

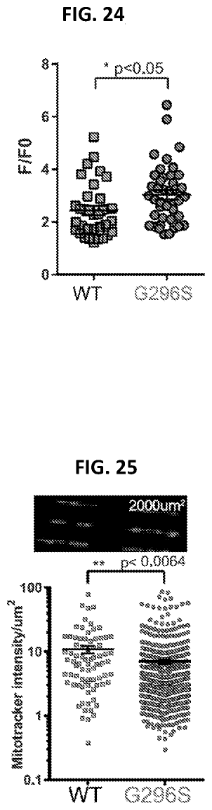

cTnT⁺ D32-cardiomyocytes were generated wildtype (wild-type), G296S, and CRISPR-corrected isogenic IPS cells (FIG. 19), although mutant lines showed slight delays in onset of spontaneous contraction (FIG. S3A-B). To more precisely model contractile properties of mature cardiomyocytes, a physiological micropatterning platform for single iPS-derived cardiomyocytes was engineered (FIG. 20) (Ribeiro et al., 2015). Only 50% of patterned G296S cardiomyocytes responded accurately to electrical pacing at 1 Hz, compared to 70% of wild-type cardiomyocytes, while wild-type cells did not respond to pacing at frequencies higher than 1 Hz, 20% of G296S cardiomyocytes were capable of beating at this faster rate (FIG. 21). G296S cardiomyocytes also had significantly reduced contractile force generation per cell movement with decreased contraction time (FIGS. 21, 22), consistent with the cardiomyopathic phenotype in patients, In patch clamp studies, G296S cells had increased overshoot potential without altered maximum upstroke velocity or action potential duration (FIG. 23), suggesting a more depolarized membrane in mutant cardiomyocytes. Calcium transients in cell clusters had increased relative peak amplitude suggesting defects in calcium ion handling (FIG. 24).

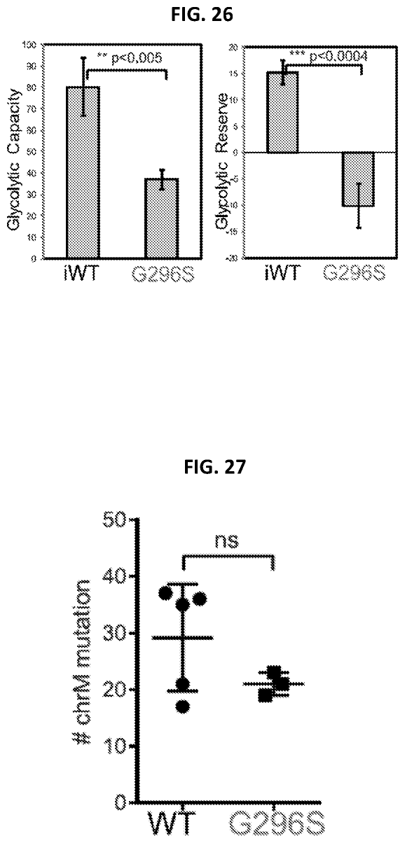

Without being bound by any particular theory, the contractile force reduction might originate from defects in mitochondrial function or metabolic activity. Indeed, single G296S cardiomyocyte on micropatterns had decreased mitochondrial staining (FIG. 25) and decreased glycolytic capacity and glycolytic reserve (FIG. 26), Although mitochondrial DNA (mtDNA) heteroplasmy has been linked to neuro-pathogenicity (Stewart and Chinnery, 2015), genome sequencing of mtDNA from G296S cells did not show increased de novo mtDNA mutations (FIG. 27). These findings demonstrate that key cardiomyocyte functions are impaired in GATA4 mutants, validating the rationale for using these patient-derived iPS cardiac cells to further dissect the mechanistic underpinnings of cardiomyopathy.

### Example 3: Attenuated Cardiac Gene Program on Mutant CPCs and Cardiomyocytes

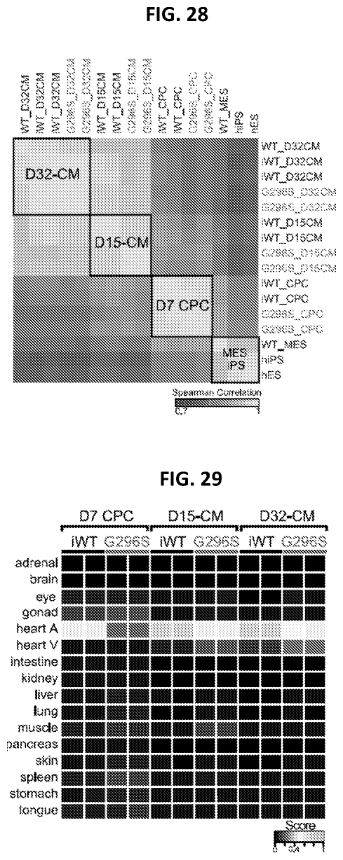

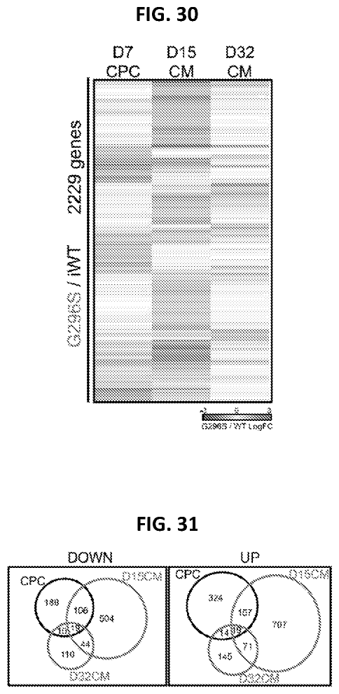

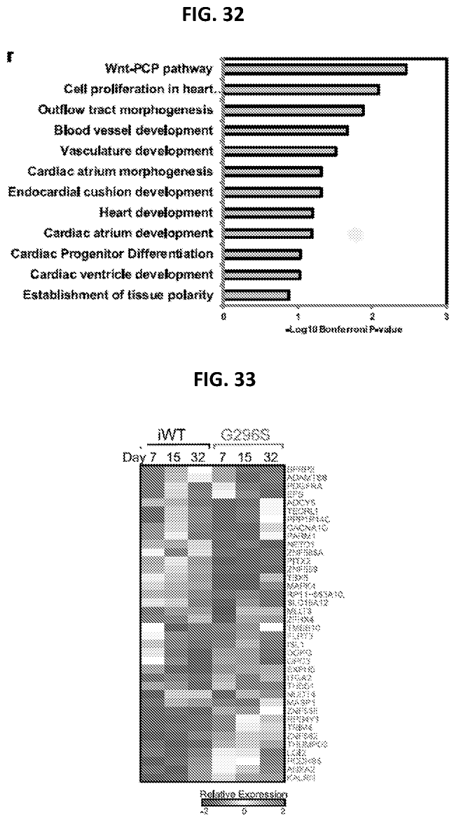

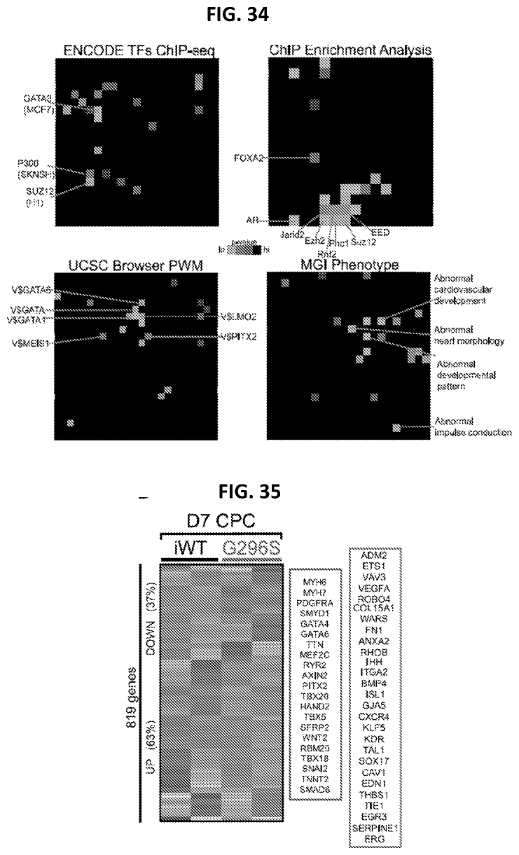

NA-seq was performed on isogenic iPS cells during cardiac differentiation into CPCs on day 7, contracting cardiomyocytes before (D15) and after (D32) lactate purification (FIG. 28). LASSO-regression algorithm (Roost et al., 2015) accurately predicted that our iWT cardiomyocyte data represented the heart transcriptome (0.6-1) rather than other human tissues, while the G296S transcriptomes from CPCs, D15-cardiomyocytes, and D32-cardiomyocytes consistently had a lower cardiac score (<0.6) (FIG. 29). 2228 genes were differentially expressed in G296S cells during at least one of the three time points with significant dynamic changes going from CPCs to mature cardiomyocytes (FIG. 30, 31). There were 38 genes involved in the Wnt-PCP pathway, or vasculature-, endocardial-, heart-development and cardiac progenitor differentiation that were consistently down- or up-regulated (FIGS. 32, 33). Network2Canves analyses (Tan et al., 2013) indicated these 38 genes were enriched for GATA-factor binding, developmentally regulated by p300 and PRC2 complex, and important for cardiovascular development and function (FIG. 34).

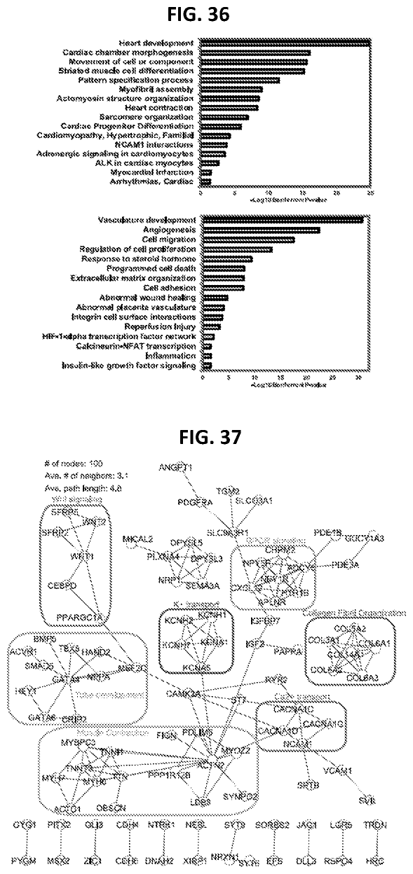

In G296S CPCs, down-regulated genes were highly connected and involved in heart development, cardiac chamber morphogenesis, myofibril assembly, heart contraction, and cardiac progenitor differentiation (e.g., MYH6, MYH7, TTN, RYR2, GATA4, GATA6, MEF2C, TBX5, and TBX18), suggesting an incomplete activation of the myocardial gene program (FIGS. 35, 36, 37). In contrast, upregulated genes were enriched for vasculature development, angiogenesis, extracellular matrix organization, integrin interactions and Calcineurin-NFAT transcription. These included TALL ETS1, ROBO4, WARS, KLF5, SOX17, TIE1, KDR and CXCR4, many of which are key signaling or transcriptional regulators of the endocardial/endothelial gene program. Percentage of CD31-posltive endothelial cells did not increase in mutant CPCs, suggesting that the upregulated endothelial/endocardial gene program was likely due to a failure in gene silencing as multi-potent CPCs adopt a cardiomyocyte fate.

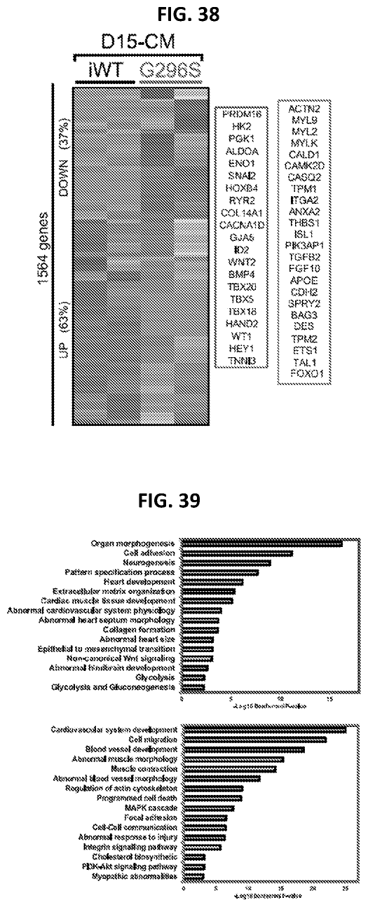

In G296S D15-cardiomyocytes, down-regulated genes were important for organ morphogenesis, heart development and glycolysis (FIGS. 38, 39). The down-regulation of glycolysis-related genes is consistent with the functional impairment in glycolytic activity. Similar to abnormalities seen at the CPC stage, up-regulated genes at D15 participated in blood vessel development, cell-cell communication, as well as Integrin and P13K-Akt signaling pathways. Persistent expression of some cardiac progenitor genes such as 1SL1, and upregulation of some smooth muscle genes, provided further evidence that abnormal activation of alterative fate genes persisted in cardiomyocytes even as they matured.

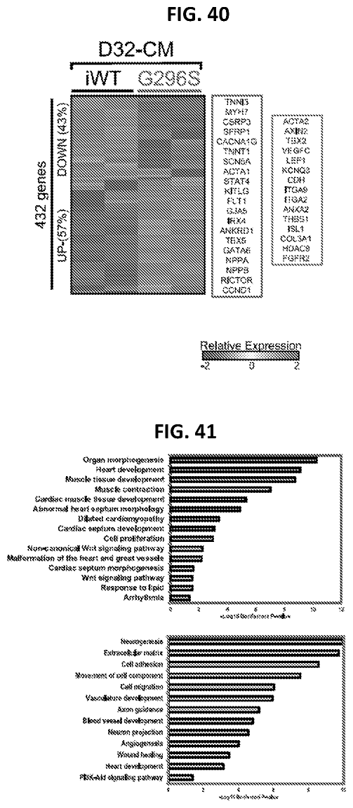

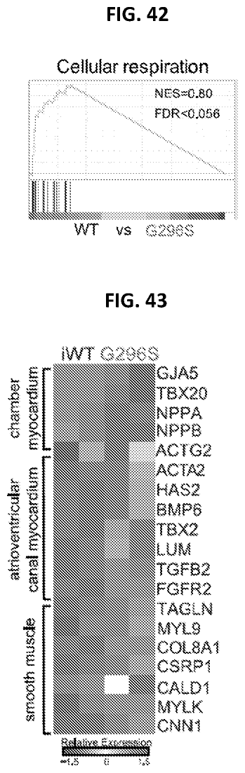

In D32-cardiomyocytes purified by lactose^{HI}glucose^{LO} media, differentially expressed genes continued to show an attenuation of the cardiac gene program and a persistent up-regulation of the endothelial/endocardial gene program (FIGS. 40, 41). Specifically, GO terms representing heart development, muscle contraction, cardiomyopathy, and cardiac septum development were enriched in downregulated genes, while up-regulated genes were enriched for vasculature development, angiogenesis and P13K-Akt signaling. Genes involved in both vascular and neuronal pathfinding were most upregulated in the neurogenesis GO category, Gene Set Enrichment Analyses (GSEA) showed reduction in cellular respiration genes in G296S cardiomyocytes (FIG. 42), consistent with the decrease in metabolic activity (FIG. 26). Furthermore, G296S cardiomyocytes down regulated chamber myocardium genes, and upregulated atrioventricular canal myocardium and smooth muscle associated genes (FIG. 43), suggesting a broader misspecification in cell identity. Upregulation of TBX2, the major transcriptional regulator of the specialized myocardium surrounding the atrioventricular valve, was notable given its function as a repressor of “working” myocardium genes and requirement for atrioventricular valve development (Aanhaanen et al., 2011). These results reveal that a single amino acid substitution in one copy of GATA4 profoundly perturbs the full acquisition and maintenance of the human cardiomyocyte gene program and provides key mechanistic explanations to the functional differences described in FIGS. 21, 23-26.

### Example 4: Open Chromatin Anomalies in GATA 4 Mutant CPCs

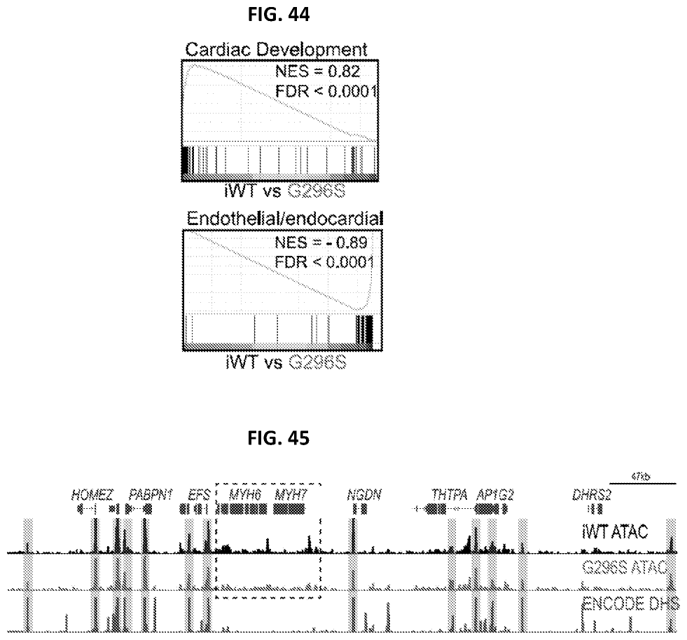

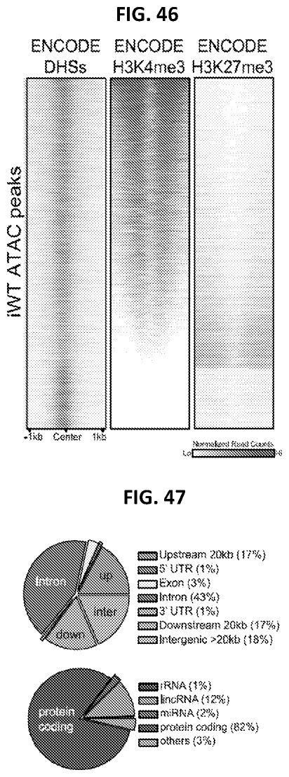

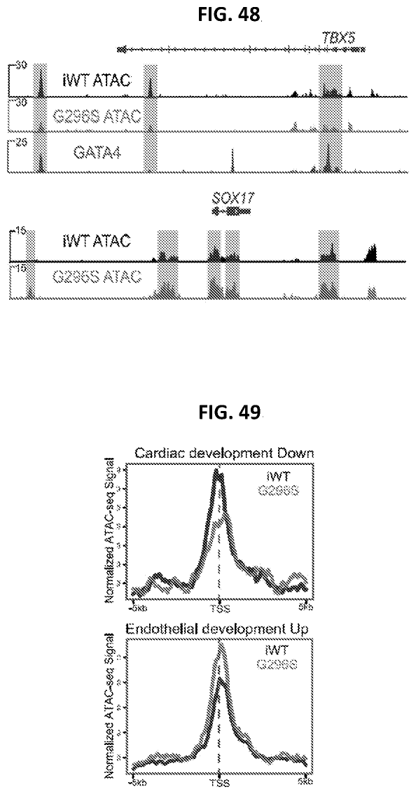

Chromatin accessibility is tightly linked to transcription factor occupancy and transcriptional output (Zaret and Carroll, 2011). To determine if genome-wide alterations in open chromatin status were responsible for the downregulation of cardiac development genes and inappropriate upregulation of endothelial/endocardial genes in G296S cells (FIG. 44), transposase accessible chromatin was analyzed by deep sequencing (ATAC-seq) in iWT and G296S CPCs (Buenrostro et al., 2013). In iWT CPCs, the 14,532 identified ATAC-seq peaks had 88% overlap with ENCODE DNase-hypersensitivity sites (DHSs) from human cardiomyocytes or ES-derived CPCs and at genomic regions expected to be transposase-accessible (FIGS. 45, 46). Furthermore, >75% had histone marks of transcriptional activation (H3K4me3) but not repression (H3K27me3), and localized mainly to introns (43%) of protein-coding genes (82%) (FIGS. 46, 47). In G296S CPCs, open chromatin status was reduced at cardiac genes as shown by a decreased ATAC-seq signal at MYH6, MYH7 (FIG. 4B, black box) and TBX5 (FIG. 48), consistent with their decreased expressions (FIG. 3D). Open chromatin status was conversely increased at S0X17, a key regulator of hemogenic-endothelium (Clarke et al., 2013). This was not idiosyncratic to a few genes as the ATAC-seq signals were coherently changed at 86-cardiac and 99-endothelial genes that were differentially expressed (FIG. 49).

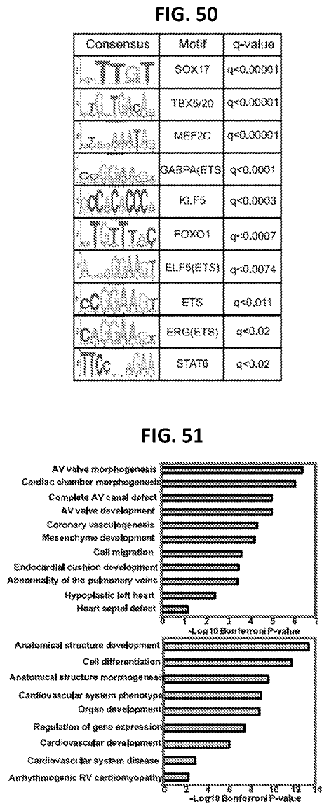

Peaks with increased ATAC-seq signal were enriched for DNA motifs of master regulators of endothelial cells (SOX17, KLF5, FOXO1, STAT6) and ETS-factors (GABPA, ELFS, ERG), providing further evidence that the endocardial/endothelial gene program failed to be silenced in G296S CPCs (FIG. 50).

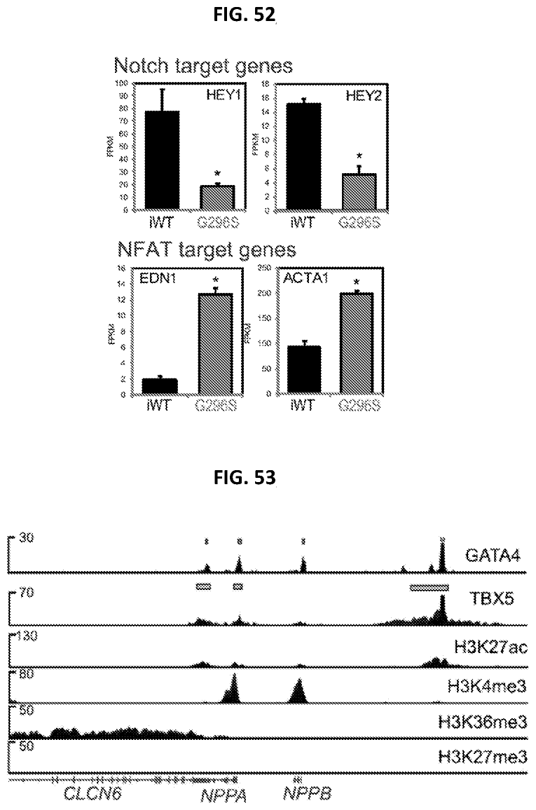

These peaks mapped to genes involved in AV valve morphogenesis, coronary vasculogenesis and endocardial cushion development (FIG. 51), consistent with the AVSD diagnosis in the individual with the GATA4 mutation (FIG. 1). Hey1 and Hey2 corepressorss are GATA4-interacting proteins (Kathiriya et al., 2004) that were down-regulated in G296S cardiomyocytes and may contribute to the failure of chromatin closure at endothelial/endocardial genes, while genes dependent on NFATc, a transcriptional regulator of the endocardium, were upregulated (FIG. 52). Thus, the incomplete cardiac gene activation and the failure to fully silence the endothelial gene program in GATA4 mutant CPCs is, in part, due to decreased chromatin accessibility at cardiac genes and increased accessibility at endothelial loci.

### Example 5: Genome-Wide Co-Occupancy of GATA4 and TBX5 I Human Cells

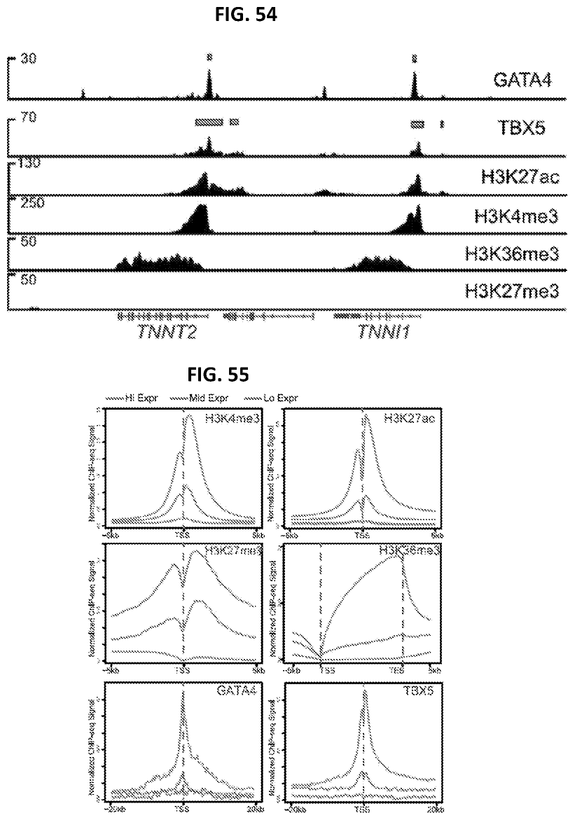

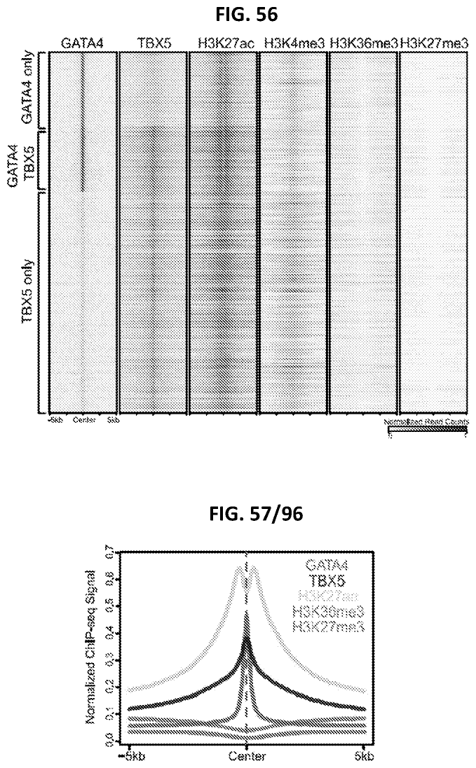

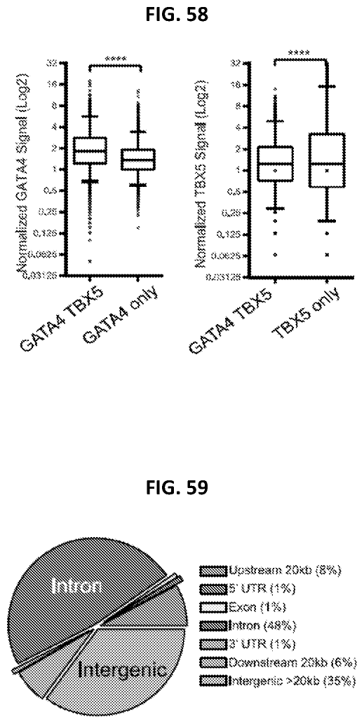

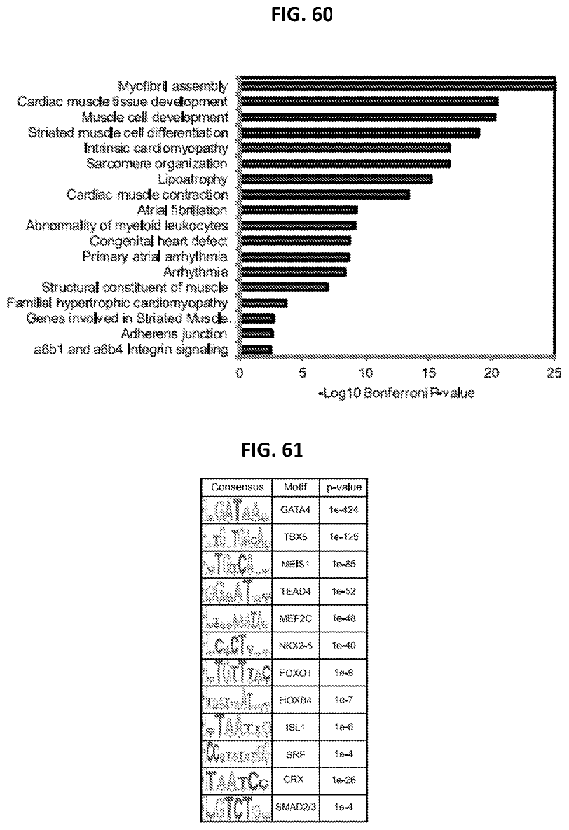

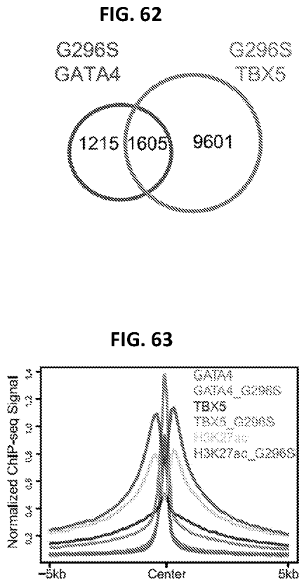

The genome-wide occupancy of GATA4 and histone marks of active promoters (H3K4me3), repressed-promoters (H3K27me3), transcription elongation (H3K36me3) and active enhancers (H3K27ac) was examined, along with the occupancy of TBX5. In wild-type cardiomyocytes, chromatin immunoprecipitation using antibodies to the endogenous protein and deep sequencing (ChIP-seq) validated many direct human targets of GATA4 and TBX5 (FIGS. 53, 54) (He et al., 2011). These gene targets were co-bound by GATA4 and TBX5 (G4T5), had high levels of H3K27ac, H3K4me3 and H3K36me3, but undetectable H3K27me3. GATA4 and TBX5 ChIP-seq signals were also positively correlated to gene expression levels (FIG. 55). Overall, GATA4, TBX5 and H3K27ac shared the strongest overlap in genome occupancy (FIG. 56), with nearly half of GATA4 sites being co-bound by TBX5 (FIGS. 56, 57). 2428 sites co-bound by human G4T5 had higher ChIP-seq signals than sites bound by GATA4 or TBX5 alone (FIG. 58). Co-bound sites mostly mapped to intronic (48%) and intergenic (35%) enhancer sites of genes for myofibril assembly, cardiac muscle development and contraction, CHD and cardiomyopathy (FIG. 59, 60). Further supporting the fidelity of the ChIP-seq, GATA4 and TBX5 motifs ranked at the top in motif analyses of G4T5 sites (FIG. 61). Motifs for TEAD4, MEF2C, NKX2.5, ISL1, SRF and SMAD2/3 were enriched, indicating a transcription factor code that maintains the cardiac gene program. Motifs near G4T5 sites were also enriched for the endothelial regulators, FOXO1 and HOXB4, suggesting a potential repressive effect of G4T5 at these sites.

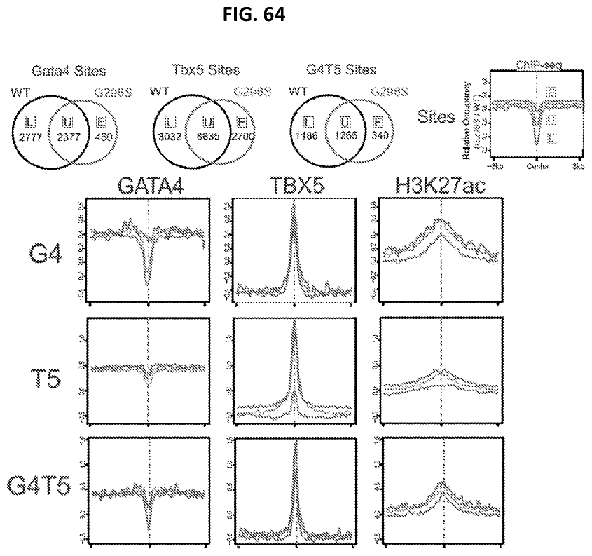

Sites bound by GATA4, TBX5 or G4T5 were systematically analyzed in G296S cells compared to wild-type (FIG. 64; top). For GATA4 sites, 54% of sites were lost (L), 46% were unchanged (U), and 16% wore ectopic sites gained (E) in mutants, suggesting dose-sensitivity for DNA binding at many sites and redistribution to others. For TBX5 sites, 26% of sites were lost (L), 74% were unchanged (U), and 24% were ectopically gained (E). G4T5 co-bound peaks were 34% less abundant in G296S than wild-type cardiomyocytes (FIG. 57, 62-63), with 48% being lost (L), 52% being unchanged (U), and 21% being gained (E).

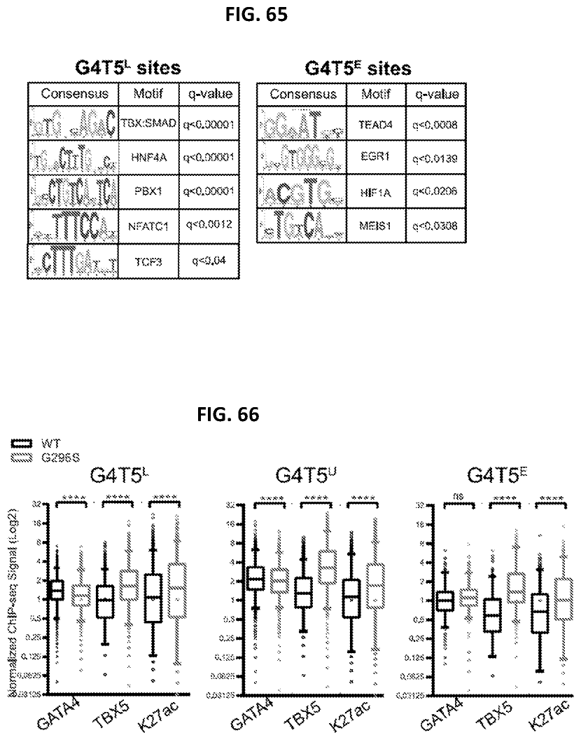

The L, U and E sites were parsed for the relative occupancy of GATA4, TBX5 and H3K27ac (FIG. 64; bottom), Consistent with the reduced DNA binding affinity of G296S GATA4, GATA4, occupancy was decreased particularly at G4^{L} and G4T5^{L} sites and correlated with increased TBX5 occupancy particularly at T5^{E} , and G4T5^{L}. A broad increase in TBX5 occupancy suggests that loss of TBX5 occupancy at other sites was unlikely due to decreased TBX5 gene expression. The active enhancer mark H3K27ac was increased most significantly at G4E, T5E, and G4T5E sites. Nearly all of the changes were statistically significant (FIG. 65) and GATA4, TBX5 and H3K27ac were not mis-localized at random genomic sites. From the RNA-seq data, genes mapping to G4T5^{L} sites were largely downregulated in CPC, 015-cardiomyocytes and 032-cardiomyocytes (FIG. 5F, S5D), Consistent with an impaired cardiac gene program, G4T5L genes were enriched in GO functions such as contractile fiber, cardiac muscle contraction, cardiac septal defect, and cardiomyopathy (FIG. 5G), Motif analyses identified TBX:SMAD, HNF4A, PBX1, NFATC1, TCF3 motifs in G4T5^{L} sites, and TEAD4, EGR1, HIF1A, MEIS1/3p-TBX5 motifs in G4T5^{E} sites (FIG. 66), many of which represent transcription factor motifs for binding partners of GATA4/TBX5 in non-cardiac lineages.

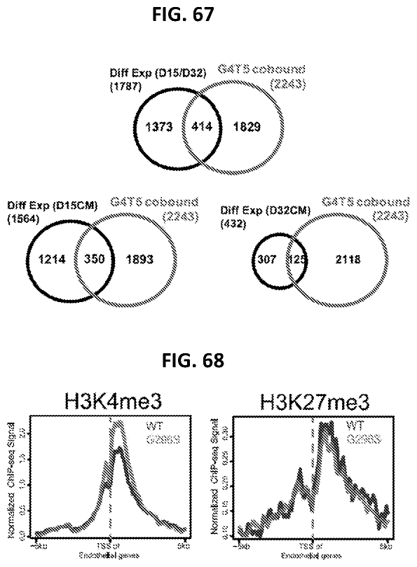

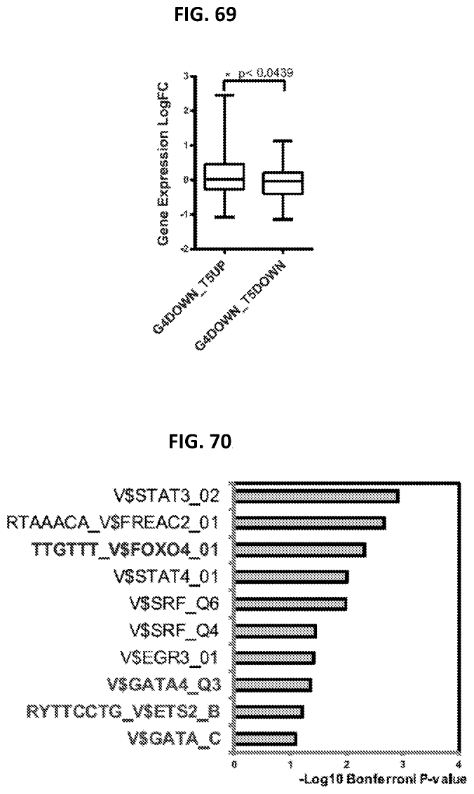

All differentially expressed genes and genes with a G4T5 site within 20 kb were examined to catalog putative direct targets of GATA4 and TBX5 in human cardiomyocytes (FIG. 67). Genes with decreased GATA4 and TBX5 binding (G4DOWN_T5DOWN) experienced a down-regulation of their expression levels (FIG. 68), suggesting that differential gene expressions were directly due to DNA binding aberrations by GATA4 and TBX5. GATA4 binding was decreased and TBX5 binding concomitantly increased at 414 putative targets (FIG. 69). GATA4 binding was reduced at 49% of sites near 82 up-regulated endothelial genes (FIG. 70). Consistent with TBX5-motif enrichment in G296S CPCs (FIG. 4G), TBX5 binding was increased at 64% of 207 TBX5 sites within these endothelial topologically associating domains, suggesting anomalous transcriptional activation by mis-localized TBX5, and possibly other coactivators. This correlated with increased H3K4me3 and decreased H3K27me3 marks at endothelial TSS in G296S cardiomyocytes (FIG. 69). Proximal promoters of up-regulated endothelial genes were also enriched in binding sites for GATA-, FOXO-, and ETS-family proteins (FIG. 70). In short, a combinatorial transcription factor binding code involving GATA4 and TBX5 is required to maintain the cardiac gene program and repress the endothelial gene program in human cardiomyocytes, and failure to properly tether TBX5 to GATA4 results in ectopic TBX5 binding and activation of the endocardial/endothelial gene program.

### Example 6: GATA4 and TBX5 Co-Regulste Human Cardiac Super-Enhancers

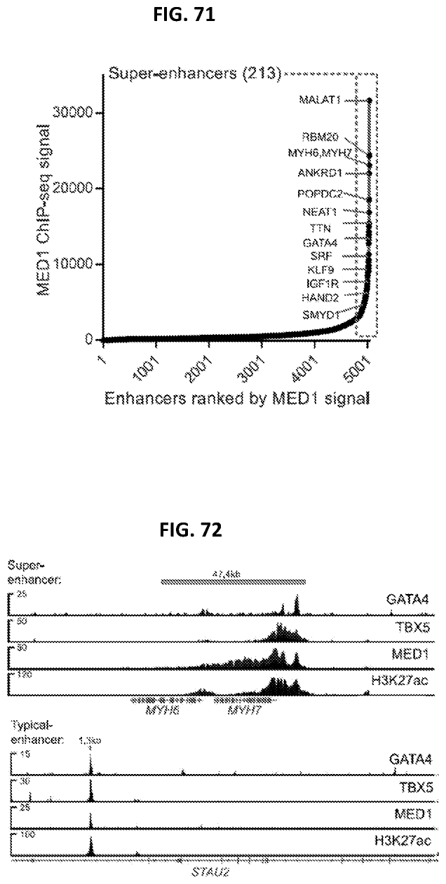

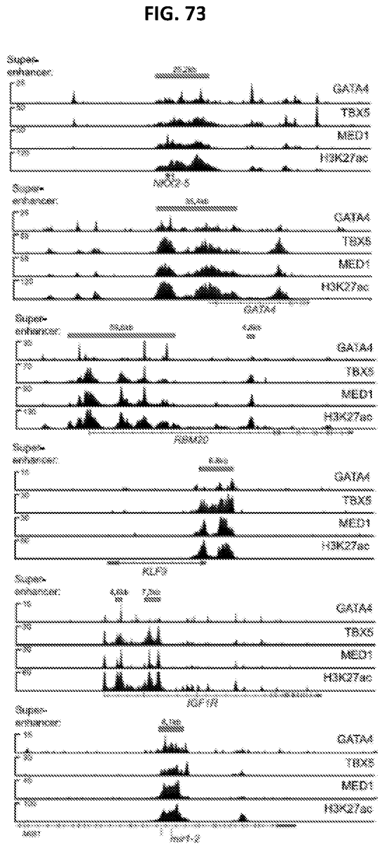

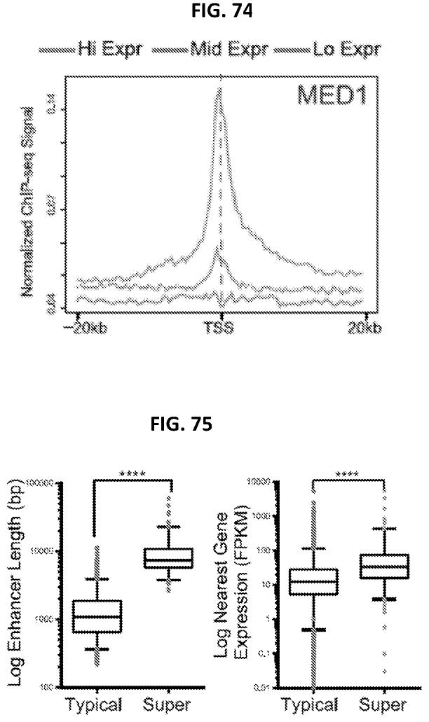

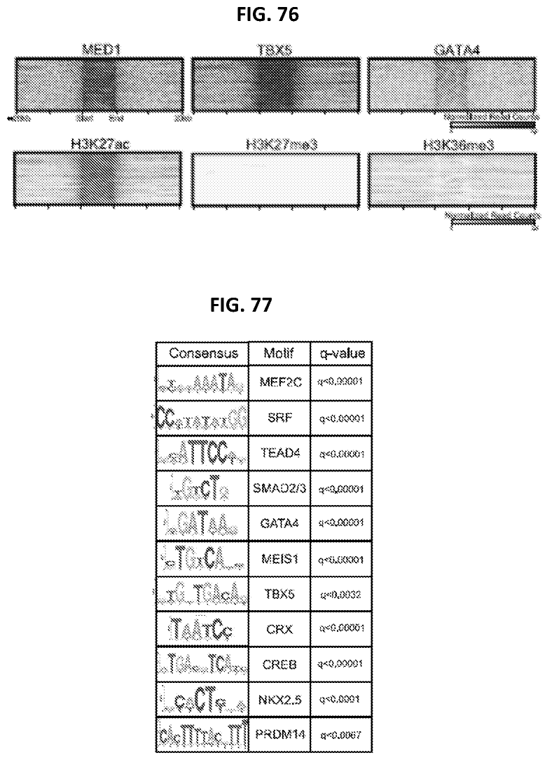

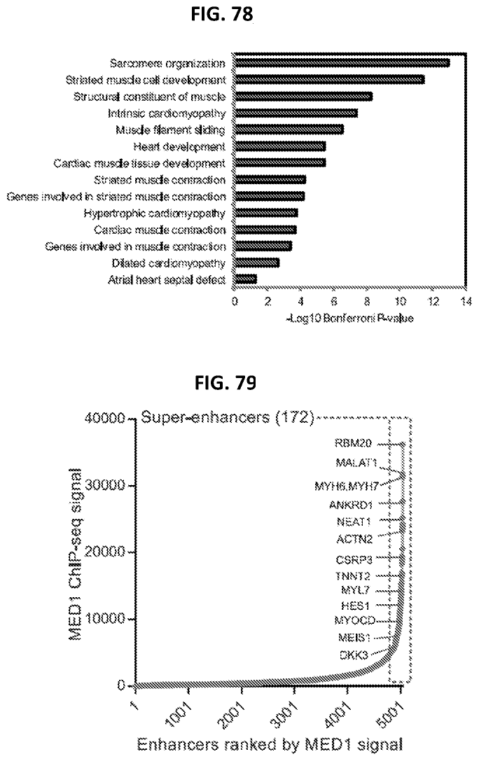

Regions of high MED1 (Mediator Complex) occupancy across several kilobases containing a high density of transcription factor motifs have been suggested to mark SEs (Whyte et al., 2013), However, MED1 classified SEs had yet to be described for human cardiomyocytes. Mis-localization of H3K27ac enrichment, another chromatin mark that has been used to identify SEs (Adam et al., 2015), in G296S mutants (FIG. 64) led to the hypothesis that the GATA4 mutation may result in altered chromatin state at SEs. 213 SEs (representing top 4%) defined by MED1 ChIP-seq In wild-type cardiomyocytes (FIG. 71). These were proximal to cardiac-enriched genes such as RBM20, MYH6/7, TTN, NKX2.5, GATA4, SRF, HAND2, miR1-2, IGF1R, with multiple constituents of G4T5 and robust H3K27ac enhancer marks (FIGS. 72, 73). MED1 ChIP-seq signal was positively correlated to gene expression levels (FIG. 74). On average, cardiac SEs had 11-fold higher MED1 binding than MED1-bound TEs, were longer (3-80 kb; average, 10 kb), and had 4-fold higher gene expression (FIG. 6C, S6C). As expected, GATA4 and TBX5 binding was strongly enriched in SE elements (FIGS. 75, 76), as were motifs for MEF2C, SRF, TEAD4, SMAD2/3, ME1S1 and NKX2.5, all critical regulators of cardiac cell fate (FIG. 77). Novel motifs included CRX, CREB and PRDM14. SE elements were near genes involved in striated muscle development. cardiomyopathy, heart development and cardiac muscle contraction (FIG. 78).

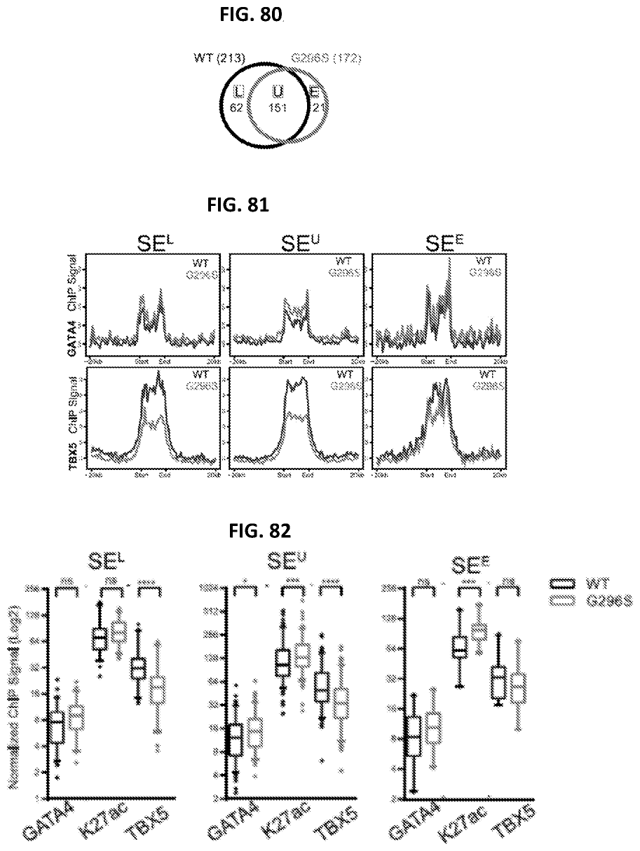

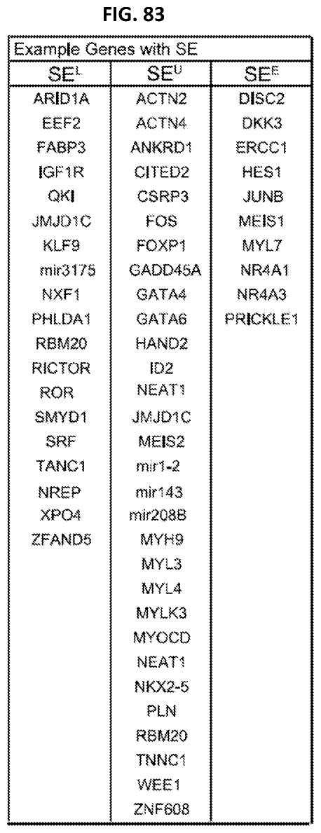

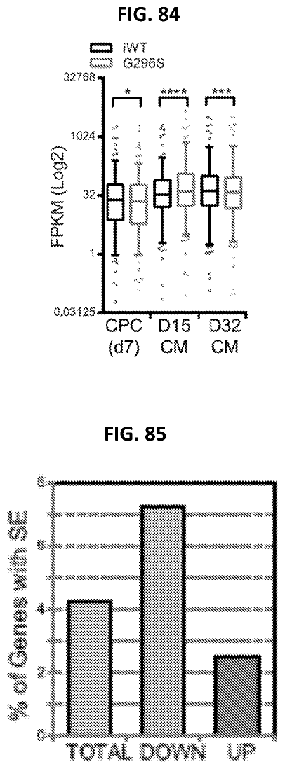

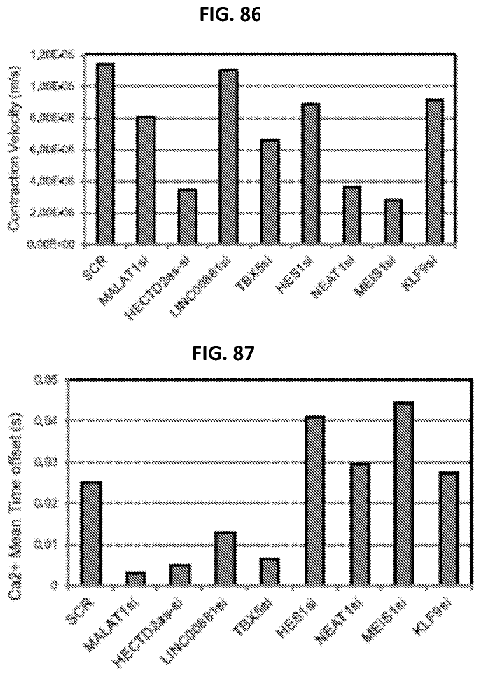

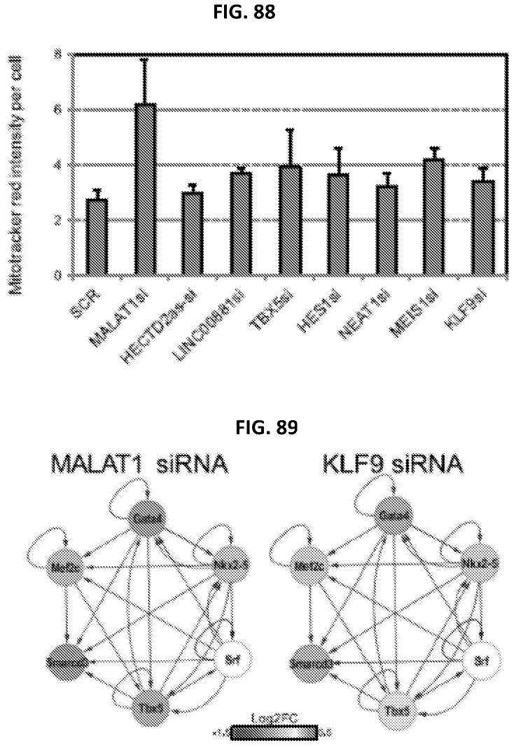

In contrast, MED1 ChIP-seq in G296S cardiomyocytes identified only 172 SE elements (FIG. 79). Comparison of SE elements showed loss of 34% (SE^{L}), with 66% being unchanged (SE^{U}) and 12% being ectopically gained (SE^{E}) in mutant cardiomyocytes (FIG. 80). TBX5 binding in the SE^{L} and SE^{U} elements were markedly reduced, despite comparable GATA4 binding (FIG. 81, 82), most likely from disruption of the GATA4-TBX5 interaction and failure of GATA4 to recruit TBX5 to cardiac SEs. Key cardiac genes with lost SE elements included IGF1R, RBM20, SMYD1 and SRF (FIG. 83), In line with a primed endothelial gene program in mutants, HES1 and JUNB gained SE elements. RNA-seq data showed altered expression levels of genes with SE elements in mutant CPCs, D15-cardiomyocytes, and D32-cardiomyocytes (FIG. 84). SE^{L} elements were enriched in MEF2A, TEAD4 and NFATC2 motifs and SEE elements were enriched in MAX:MYC, MEIS1 and GATA4 motifs (Figure S6F). Down-regulated genes from the RNA-seq data were disproportionally enriched for SE elements (FIG. 85). In addition, SE elements mapped to several long-non-coding RNAs (MALAT1, HECTD2as, LIN00881, NEAT1), and transcription factors (HES1, KFLF9) with undetermined cardiogenic functions. Their knockdown in cardiomyocytes mostly induced abnormalities in contractility, calcium flux, and mitochondria mass (FIGS. 86-88). Depletion of MALAT1 and KLF9 further induced a collapse of the core cardiac transcriptional network (FIG. 89) (He et al., 2011). These data show that the predicted SE elements may play critical cardiac functions and their mis-regulation is an important consequence of GATA4 mutation.

### Example 7: Regulatory Hubs in a GATA4-TBX5 Network Centered on PI3K Signaling

A systems-biology approach using genome-wide maps of transcription factor localizations, mediator enrichment and transcript abundance were used to elucidate the gene regulatory networks disrupted more broadly. To construct a GATA4-TBX5 controlled gene regulatory network, downregulated and upregulated genes in G296S engineered cardiomyocytes, G4T5 bound genes in wild-type or G296S cardiomyocytes, and genes with super enhancer elements were integrated with STRING datasets as shown below:

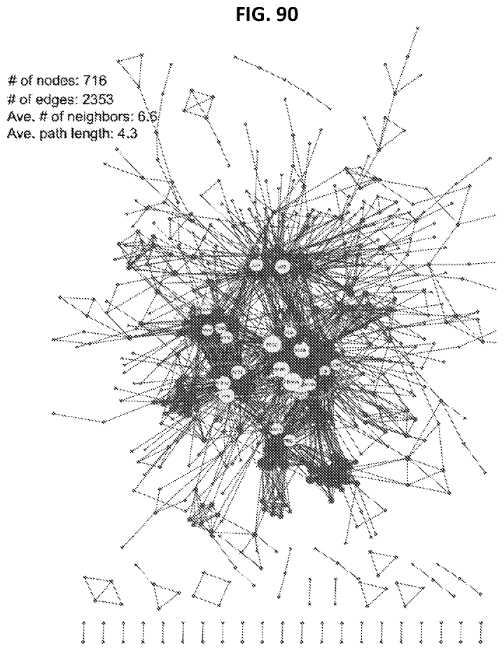

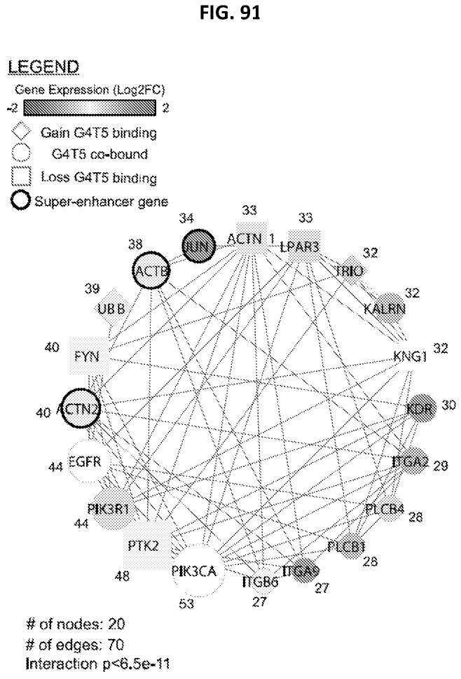

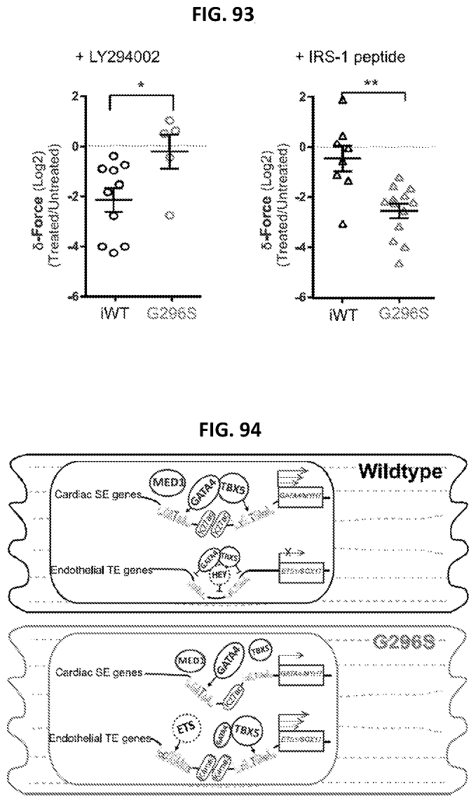

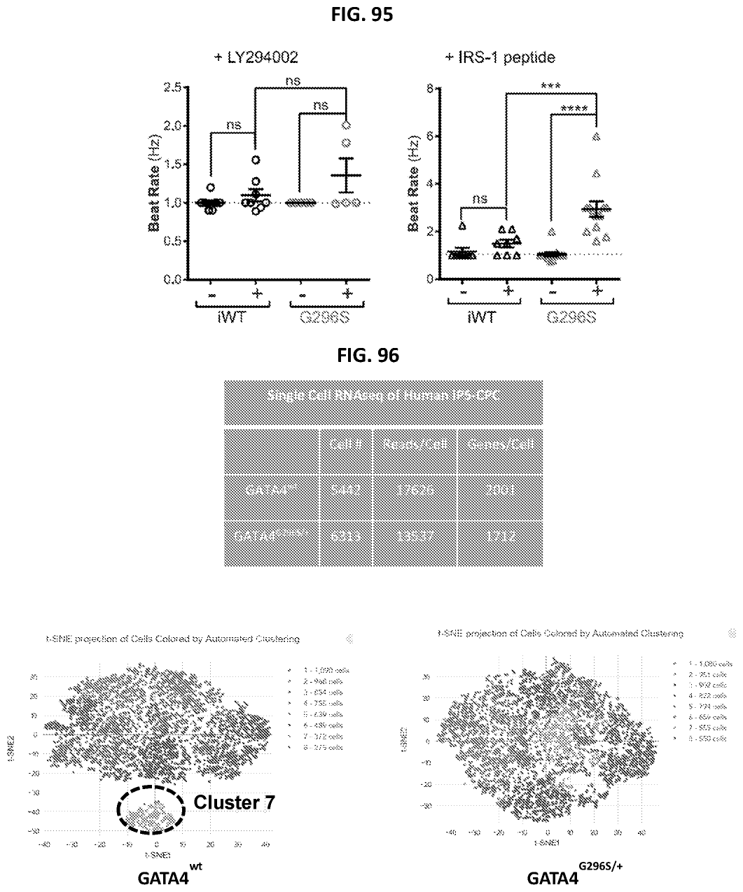

A “scale-free” network (Barabasl and Albert, ⋅1999) of 716 nodes connected by 2,353 edges with an average 6.6 neighbors and path length of 4.3 (FIG. 90). Nodes were connected by edges representing physical (protein-protein) or functional (genetic, co-expression, co-occurrence) interactions. At least 5 sub-networks connected through 20 regulatory “hubs” were identified. The top-20 hubs were extracted as a sub-network connected by 70 edges, and each had 27-53 neighbors—a 4 to 8-fold more than the average node in the gene regulatory network (FIG. 91). This sub-network had a statistically significant interaction of p<6.5 e⁻¹¹. Interestingly, the top-4 hubs were G4T5 co-bound genes linked to PI3K signaling: PIK3CA (α-catalytic subunit), P1K3R1 (regulatory subunit), and PTK2 and EGFR, the upstream signal transduction components. In PTK2, G4T5 co-occupancy was lost in GATA4 mutants (FIG. 91). ITGA2, 1TGA9 and KDR were also hubs and involved In P13K signaling. Gene ontology analysis showed significant enrichment for integrin, P13K-Akt, Phosphatidylinositol and EGF signaling with a net predicted increase in P13K signaling (FIG. 92). Importantly, when engineered cardiomyocytes were treated with a PI3K inhibitor (LY294002), iWT cardiomyocytes displayed a significant decrease in force generation, but G296S cardiomyocyte were insensitive or had an increase in force production (FIG. 93). Conversely, treatment with an insulin receptor substrate (IRS) synthetic peptide to activate P13K signaling dramatically reduced force generation in G296S cardiomyocytes (FIG. 93, right). Although the P13K inhibitor had no effect on beat rates in wild-type cells, the IRS peptide increased beat rates in G296S cardiomyocytes 3-fold greater than iWT cardiomyocytes, consistent with a hyper-sensitivity to P13K pathway activation (FIGS. 93-95). Cardiac gene loci have reduced open chromatin and TBX5 binding to SE elements which reduces transcription; aberrantly open chromatin is enriched for TBX5, along with motifs for ETS factors and other endothelial regulators resulting in failure to silence transcription at endothelial genes and other sites (FIG. 95).

Without being bound by any mechanistic theory, the empirical evidence provided herein shows that the current proper cardiac development and function require an open chromatin signature for GATA4-TBX5 co-occupancy in MED1 bound, H3K27ac-marked SE elements to activate transcription of key cardiogenic genes (FIG. 94; top), In GATA4 heterozygosity with a missense mutation that affects protein-protein interactions, a loss of TBX5 co-occupancy in Med1-bound, H3K27ac-marked SE elements to activate transcription of key cardiac genes (FIG. 94, top). TBX5 recruitment to SE elements is associated with failure to maintain open chromatin and diminished transcription of key cardiac genes (FIG. 94; bottom). The GATA4 G296S mutation is associated with mis-localization of TBX5, and possibly other transcriptional activators (e.g. ETS-factors), resulting in an open chromatin signature at endothelial promoters leading to inappropriate endothelial gene expression and aberrant activation of alternative lineages. These studies demonstrate how TF complexes cooperatively regulate genome wide localization of transacting factors to precisely control activation and repression of gene expression, and how this can be disrupted by human disease-causing mutations.

In short, the experimental evidence that GATA4 mutant cardiomyocytes exhibit dysregulated PI3K signaling is consistent with this aspect of the computationally predicted network and provides a potential node for partially correcting the diseased GATA4-TBX5 gene regulatory network.

### Example 8: T-SNE Plot of Single Cell RNA-SEQ of Human IPS-CPC

Both WT and G296S^{+/-} cells were subject to single cell expression analysis using RNA-seq. A t-SNE plot was generated for the data using a Seurat software package. See, e.g., BMC Genomics. 2013, 14:302.DOI: 10.1186/1471-2164-14-302. The t-SNE plot allowed visualization of the RNA-seq single cell data by giving each datapoint a location in a two or three-dimensional map. The results of this data are set forth in FIG. 96.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. All references cited herein are incorporated by their entirety for all purposes. In the claims that follow, unless the term “means” is used, none of the features or elements recited therein should be construed as means-plus-function limitations pursuant to 35 U.S.C. § 112, 16.

1. A population of engineered cardiomyocytes wherein the cells are generated from mammalian pluripotent stem cells, and wherein the cells comprise at least one mutation associated with an in vivo cardiac phenotype.

2. The population of claim 1, wherein the in vivo cardiac phenotype is cardiomyopathy.

3. The population of claim 1, wherein the cells are generated from human pluripotent stem cells.

4. The population of claim 1, wherein the cells are generated from human induced pluripotent stem cells.

5. The population of claim 4, wherein the induced pluripotent stem cell is derived from a subject with cardiomyopathy.

6. The population of claim 1, wherein the cells comprise one or more mutations that cause disruption of GATA4 and/or TBX5 binding at super enhancer elements associated with genes required for heart development and/or muscle contraction.

7. The population of claim 1, wherein the engineered cardiomyocytes comprise at least one mutation in the PI3K pathway.

8. The population of claim 1, wherein the engineered cardiomyocytes comprise at least one mutation in the GATA4 gene.

9. The population of claim 1, wherein the engineered cardiomyocytes comprise at least one mutation that inhibits the interaction between GATA4 and TBX5.

10. An engineered mammalian embryonic stem cell comprising at least one mutation that causes disruption of GATA4 and/or TBX5 binding at super enhancer elements associated with genes required for heart development and/or muscle contraction.

11. The embryonic stem cell of claim 10, wherein the stem cell is rodent.

12. The embryonic stem cell of claim 10, wherein the stem cell is human.

13. A method for increasing force generation or increasing beat rate in an engineered cardiomyocyte, comprising administering to the cell an activator of PI3K.

14. The method of claim 13, the engineered cardiomyocyte having a mutation in the GATA4 gene.

15. A method for screening a candidate agent, the method comprising:
contacting the candidate agent with an engineered cardiomyocyte in vitro, wherein (i) the engineered cardiomyocyte comprises at least one mutation that causes disruption of GATA4 and/or TBX5 binding at super enhancer elements associated with genes required for heart development or muscle contraction; or (ii) the engineered cardiomyocyte comprises at least one mutation the engineered cardiomyocytes comprise at least one mutation in the PI3K pathway; and
using an in vitro assay to detect an effect of the candidate agent on the phenotype of the engineered cardiomyocytes.

16. The method of claim 15, wherein the engineered cardiomyocytes comprises at least one mutation that inhibits the interaction between GATA4 and TBX5.

17. The method of claim 15, wherein the engineered cardiomyocytes comprises at least one mutation in the GATA4 gene.

18. (canceled)
